(19) United States
(12) Patent Application Publication
(10) Pub. No.: US 2007/0258952 A1
(43) Pub. Date: Nov. 8, 2007

(54) Anti-Tumor Activity of an Oncolytic Adenovirus-Delivered Oncogene siRNA

(75) Inventors: Alex W. Tong, Dallas, TX (US); Yu An Zhang, Plano, TX (US); John J. Nemunaitis, Dallas, TX (US); Yuqiao Shen, Orinda, CA (US); Po-hsun Chen, Addison, TX (US); Shirley K. Samuel, Irving, TX (US)

Correspondence Address: CHALKER FLORES, LLP, 2711 LBJ FRWY, Suite 1036, DALLAS, TX, 75234, US

(73) Assignees: BAYLOR RESEARCH INSTITUTE, Dallas, TX (US); MUREX PHARMACEUTICALS, Dallas, TX (US); BAYLOR UNIVERSITY, Waco, TX (US)

(21) Appl. No.: 11/744,236

(22) Filed: May 4, 2007

Related U.S. Application Data
(60) Provisional application No. 60/797,918, filed on May 4, 2006.

(51) Int. Cl. A61K 48/00; C12N 15/861

(52) U.S. Cl. 424/93.2; 435/456

(57) ABSTRACT

The present invention includes compositions and methods for the knockdown of one or more genes to a target cell in need of gene therapy by using an siRNA transgene that is integrated into a replication-competent, oncolytic adenovirus.

### CROSS-REFERENCE TO RELATED APPLICATIONS

This application claims priority to U.S. Provisional Patent Application Ser. No. 60/797,918, filed May 4, 2007, the entire contents of which are incorporated herein by reference.

### TECHNICAL FIELD OF THE INVENTION

The present invention relates in general to the field of viruses having anti-tumor activity, and more particularly, to compositions and methods for delivering siRNA to target cells with high efficiency.

### BACKGROUND OF THE INVENTION

Without limiting the scope of the invention, its background is described in connection with gene delivery.

A number of post-transcriptional gene expression suppression mechanisms occur in a wide variety of organism, e.g., interfering RNA (siRNA); a micro, interfering RNA (miRNA); a small, temporal RNA (stRNA); short, hairpin RNA (shRNA); small, interfering DNA (siDNA); or even a short, hairpin DNA (shDNA). The phenomenon known generally as RNA interference (RNAi), originally referred to as “cosuppression” by Napoli and coworkers (4), involves highly potent, sequence-specific, post-transcriptional gene silencing (PTGS) by double-stranded RNA (dsRNA). In plants and insects, RNA interference is mediated by 21-23 nucleotide siRNAs that are derived from long precursors through cleavage by Dicer, an endogenous RNase III-like protein (reviewed in 5). Tuschl and coworkers subsequently validated RNAi activity in mammalian cells by introducing short, synthetic siRNA duplexes to attain sequence-specific RNA-knockdown (1).

Cleavage of the mRNA target involves the participation of a self-aggregating cytoplasmic protein complex called the “RNA interfering silencing complex” (RISC) having an intrinsic, ATP-dependent helicase activity that unravels the duplex siRNA. Through Watson-Crick base pairing with the target mRNA sequence, the RISC-bound, antisense strand of the siRNA initiates ATP-independent cleavage by “Slicer”, a member of Argonaute family of endonucleases (5). Systemically introduced siRNAs displayed similar pharmacokinetics as antisense oligonucleotides (ASOs). However, markedly extended silencing activity of up to 22 days have been demonstrated after the siRNA gains entry intracellularly, apparently through siRNA stabilization through complexing with RISC and/or available, uncleaved target mRNA (6). This feature, together with the potency and target specificity of siRNAs, offers the promise of a higher therapeutic index as compared with ASOs or antibody/small molecule-based therapeutics (2).

siRNA knockdown of the mutant K-ras oncogene, one of the most common oncogenetic mutations in human cancers, has generated pronounced anti-tumor effects (7). When delivered as a non-replicative retroviral transgene, siRNA^{ras} inhibited the relevant mutant K-ras^{v12} allele and collaterally abrogated anchor independent growth and tumorigenicity (8). Similar anti-tumor activities were also attained in vivo through siRNA knockdown of other critical components for tumor cell growth, metastasis, angiogenesis, and chemoresistance (reviewed in 5). While these findings suggest that siRNAs may serve as a novel and effective class of tumor therapeutics through PTGS, efficient, in vivo delivery of active siRNA remains a technical challenge.

A key barrier for siRNAs to attain clinical efficacy has been the lack of an optimal delivery platform. siRNA has been administered effectively in mice through hydrodynamic injection (9), however, hydrodynamic injection is not feasible clinically due to potentially life-threatening hypotension and transient heart failure events (2). Consistency of transgene activity and potential toxicity at therapeutic doses remain to be concerns and challenges for cationic liposome-based delivery (10). Stable transfection and expression of siRNA has been attained through delivery by non-replicating viruses at the locoregional level (11,12), however, this approach limits target cell coverage to the initial infectious event.

On patent in this area is U.S. Pat. No. 7,022,828 issued to McSwiggen for the use of siRNA in the treatment of diseases or conditions related to levels of IKK-gamma. Specifically, this group uses nucleic acid molecules, including antisense and enzymatic nucleic acid molecules, such as hammerhead ribozymes, DNAzymes, allozymes, aptamers, decoys and siRNA (RNAi), which modulate the expression or function of IKK genes, such as IKK-γ, IKK-α, or IKK-β, and PKR genes. However, the issue of efficient delivery of the siRNA in a clinically relevant manner limits the use of the technology.

### SUMMARY OF THE INVENTION

The present invention relates to a gene delivery vector that includes a replication-competent, oncolytic adenovirus that expresses a siRNA. The target cell for the vector may be a human cancer cell, e.g., in vivo. The adenovirus may be any of the known oncolytic adenovirus an ONYX virus, e.g., an ONYX-411, ONYX-200 or an ONYX-015 virus, or the most commonly used Ad5Δ24. The siRNA may target a gene that encodes, e.g., an oncogene, a transcription factor, a receptor, an enzyme, a structural protein, a cytokine, a cytokine receptor, a lectin, a selectin, an immunoglobulin, a kinase and a phosphatase. Specific examples of siRNA targets may include, e.g., siRNA vs. p53, p16, p21, MMAC1, p73, zac1, C-CAM, BRCA1, Rb, Harakiri, Ad E1 B, ICE-CED3 protease, IL-2, IL-3, IL-4, IL-5, IL-6, IL-7, IL-8, IL-9, IL-10, IL-11, IL-12, IL-13, IL-14, IL-15, TNF, GMCSF, α-interferon, β-interferon and γ-interferon, CFTR, EGFR, VEGFR, IL-2 receptor and the estrogen receptor, Bcl-2 gene family (Bcl-2 or Bcl-xL), ras, myc, neu, raf erb, src, fins, jun, trk, ret, gsp, hst, and abl. For example, the siRNA may target a K-Ras oncogene and kills at least 35% of the target cells. The siRNA may be an oncogene K-ras^{v12}-specific siRNA^{ras-4} hairpin insert.

Other examples include siRNAs for target genes that express, e.g., amyloid protein, amyloid precursor protein, angiostatin, endostatin, METH-1, METH-2, Factor IX, Factor VIII, collagen, cyclin dependent kinase, cyclin D1, cyclin E, WAF 1, cdk4 inhibitor, MTS1, cystic fibrosis transmembrane conductance regulator, IL-1, IL-2, IL-3, IL-4, IL-5, IL-6, IL-7, IL-8, IL-9, IL-10, IL-11, IL-12, IL-13, IL-14, IL-15, IL-16, IL-17, erythropoietin, G-CSF, GM-CSF, M-CSF, SCF, thrombopoietin, BNDF, BMP, GGRP, EGF, FGF, GDNF, GGF, HGF, IGF-1, IGF-2, KGF, myotrophin, NGF, OSM, PDGF, somatotrophin, TGF-β, TGF-α, VEGF, interferon, TNF-.alpha., TNF-.beta., cathepsin K, cytochrome p-450, farnesyl transferase, glutathione-s transferase, heparanase, HMG CoA synthetase, n-acetyltransferase, phenylalanine hydroxylase, phosphodiesterase, ras carboxyl-terminal protease, telomerase, TNF converting enzyme, E-cadherin, N-cadherin, selectin, CD40, 5-α reductase, atrial natriuretic factor, calcitonin, corticotrophin releasing factor, glucagon, gonadotropin, gonadotropin releasing hormone, growth hormone, growth hormone releasing factor, somatotropin, insulin, leptin, luteinizing hormone, luteinizing hormone releasing hormone, parathyroid hormone, thyroid hormone, thyroid stimulating hormone, immunoglobulin, CTLA4, hemagglutinin, major histocompatibility factor (MHC), VLA-4, kallikrein-kininogen-kinin system, CD4, sis, hst, ras, abl, mos, myc, fos, jun, H-ras, ki-ras, c-fins, bcl-2, L-myc, c-myc, gip, gsp, HER-2, bombesin receptor, estrogen receptor, GABA receptor, EGFR, PDGFR, FGFR, NGFR, GTP-binding regulatory proteins, interleukin receptors, ion channel receptors, leukotriene receptor antagonists, lipoprotein receptors, opioid pain receptors, substance P receptors, retinoic acid and retinoid receptors, steroid receptors, T-cell receptors, thyroid hormone receptors, TNF receptors, tissue plasminogen activator; transmembrane receptors, calcium pump, proton pump, Na/Ca exchanger, MRP 1, MRP2, P170, LRP, cMOAT, transferrin, APC, brca1, brca2, DCC, MCC, MTS1, NF1, NF2, nm23, p53 and Rb.

Another embodiment of the present invention is a vector that includes a replication-competent, oncolytic adenovirus with an siRNA insert that when expressed mediates siRNA-mediated oncogene knockdown and viral oncolysis. The present invention also includes a method of modulating gene expression by contacting a target cell with a vector that is a replication-competent, oncolytic adenovirus with an siRNA expression insert that mediates siRNA-mediated oncogene knockdown and viral oncolysis.

Yet another embodiment of the present invention includes a method of treating a patient in need of gene therapy by identifying one or more target cells that are in need of gene therapy; making a replication-competent, oncolytic adenovirus with an siRNA that mediates siRNA-mediated gene modulation and contacting the target cell with the adenovirus.

The small interfering RNAs (siRNA) of the present invention are generally small double stranded RNA molecules that mediate specific and highly potent post-transcriptional gene modulation. The present invention overcomes the challenges with the delivery of siRNA by placing the siRNA gene under the control of a conditional promoter in an optimized delivery platform. The present inventors determined the applicability of the replication-competent, oncolytic adenovirus ONYX-411 to deliver a mutant K-ras siRNA transgene to human cancer cells. The K-ras^{v12}-specific siRNA^{ras-4} hairpin construct under control of the human H1 promoter was cloned into the deleted viral E3B region for coordinate expression with late viral genes. This novel construct (Internavec, for interfering RNA vector) acquired an approximately 10-fold increase in potency (as compared with the parental virus) against human cancer cells expressing the relevant K-ras^{v12} mutation. Internavec remained attenuated in the human normal epithelial line HMEC, suggesting that this delivery platform may serve to limit any potential “off target” siRNA effects (3) to viral permissive cancer cells.

In one example, intratumoral injections of Internavec (5 daily injections of 1×10⁸ pfu) completely inhibited the growth of H79 human pancreatic cancer xenografts in 3 of 5 mice, and were significantly more effective than treatment with parental virus or the control siRNA construct ONYX-411-siRNA^{GFP}. Further analysis demonstrated that siRNA^{ras} transgene activity contributed to cell cycle blockage, increased apoptosis and enhanced tumor cytotoxicity. Thus, the present invention is a two-pronged attack on tumor cells through oncogene knockdown and viral oncolysis, resulting in a significantly enhanced antitumor outcome.

The present inventors recognized a need for the use of oncolytic virus platforms as a marked improvement from targeted siRNA transgene delivery using non-replicative viral delivery. Through early viral gene deletions and/or substitutions, oncolytic DNA viruses display restricted viral replicative activity in viral permissive cancer cells with inherent tumor suppressor gene defects (p53, pRb) or overexpressed transcription factors (E2F-1)(13,14,15).

The siRNA delivery by an oncolytic virus of the present invention will find wide-spread use for several reasons. First, tumor-selective infectivity implies that the viral-delivery vehicle restricts transgene expression to the cancer microenvironment, hence minimizing potential cytotoxicity to normal tissues (carrier-defined specificity). Second, transgene expression is extended through viral replication and reinfection of permissive cancer cells. Further, the viral oncolytic process is expected to augment anti-tumor outcomes of siRNA-mediated knockdown of the cancer genetic apparatus. The replication-competent oncolytic adenovirus, as exemplified by dl1520 (ONYX-015), is well characterized clinically with respect to high infectivity and safety (15). Therapeutic doses (up to 10¹² viral particles) of dl1520 and other oncolytic adenoviruses are well tolerated intratumorally and intra-arterially in over 500 patients (reviewed in 15), and have produced clinical efficacy at the locoregional level in advanced head and neck cancer, pancreatic carcinoma, and metastatic colorectal carcinomas (15,16,17).

### BRIEF DESCRIPTION OF THE DRAWINGS

For a more complete understanding of the features and advantages of the present invention, reference is now made to the detailed description of the invention along with the accompanying figures and in which:

FIGS. 1a to 1d summarize the anti-tumor activities of siRNA^{ras};

FIG. 1a. Diagrammatic illustration of K-ras siRNA constructs. 3 siRNA constructs targeting K-ras exon 1 region were designed as siRNA^{ras-2} (targeting consensus global wt sequence), siRNA^{ras-4} (targeting K-12 GTT/v12 mutation sequence), siRNA^{ras-5} (targeting K-12 wt/g12sequence);

FIG. 1b. K-ras siRNAs effectively down-regulated K-ras mRNA expression. H441 cells were liposome-transfected with siRNA oligonucleotides (150 nM, 22 hrs). Total RNA was extracted from untreated or siRNA-transfected, and K-ras mRNA expression was quantified by a one-step RT-PCR reaction with co-amplification of the internal control β-actin gene. Data represents mean±SD (n=3);

FIG. 1c. Anti-proliferative activity of K-ras siRNA. Proliferative activity of untreated cultures of H441 cells was compared with their mock-transfected (transfection reagent only) or siRNA oligonucleotide-transfected (150 nM, 72 hrs) counterparts by a bromodeoxyuridine (BrdU) labeling assay. Value represents mean±SD (n=3);

FIG. 1d. qPCR quantification of ONYX-411. A549 cells (7.5×10⁴) were infected ONYX-411 for 24 hrs at 5 MOI with or without subsequent transfection with siRNA^{ras-2} (150 nM). Total viral DNA yield was determined by qPCR quantification of total DNA isolated from A549 cell pellets, using the primers specific to adenovirus 5 hexon region;

FIG. 1e. Combined anti-tumor activities of ONYX-411 and K-ras siRNA. Log growth phase A549 cultures were infected with ONYX-411 (5 MOI) only at time 0, or followed by transfection siRNA^{ras-2} (150 nM) at 24 hrs post-viral infection. Viable cells were enumerated by trypan blue exclusion analysis;

FIGS. 2a to 2g summarize the in vitro Properties of the siRNA vector system referred to herein as Internavec;

FIG. 2a. Illustration of the Internavec construct. Internavec was generated by cloning the human H1-RNA promoter driven, K-ras^{v12} oncogene reactive siRNA hairpin sequence into the E3B region of ONYX-411, an E2F/Rb regulated replicative-competent adenovirus;

FIG. 2b. Down-regulation of K-ras mRNA by Internavec treatment. H79 cells were infected with Internavec or control ONYX-411 (5 MOI) for 48 hrs prior to concomitant amplification of K-ras mRNA and internal control β-actin mRNA by one-step RT-PCR (see Methods). The level of K-ras mRNA expression of each sample was normalized to β-actin mRNA expression;

FIG. 2c. Enhanced growth inhibitory activity of Internavec. The growth inhibitory effect of Internavec () was compared with that by ONYX-411 (▪) (5 MOI) by trypan blue exclusion quantification of viable cells. Values were normalized and represented as % of untreated culture;

FIG. 2d. Relative cytotoxic activity of Internavec () vs. ONYX-411 (▪) and ONYX-411-siRNA^{GFP} (♦). Relative cytotoxic activity was compared by determining the ED₅₀ of the three viral constructs, i.e. effective dose required to generate a 50% cytotoxic response in H79 cells by the MTT assay. H79 cells were infected with Internavec, control ONYX-411 or ONYX-411-siRNA^{GFP} at a dose range of 1 to 25 MOI for 120 hrs prior to standard MTT analyses. Mean ±SD was given in each data point. *: p<0.05; **: p<0.01;

FIG. 2e. Anti-tumor activity of Internavec in human cancer lines with or without the relevant K-ras mutation. MTT analysis was carried out with cancer lines with the relevant K-ras mutation (K-ras^{v12}; H79, SW480, H441) or a wt K-ras (K-ras^{g12}) phenotype (H522, H596). Data represents parallel analyses of cultures treated with Internavec or ONYX-411 at 5 MOI, except for SW480 cells that were treated at 1 MOI due to high sensitivity of this cell line to adenoviral lysis at higher MOIs. *: p<0.05; ***: p<0.001; ****: p<0.0001;

FIG. 2f & FIG. 2g. Viral yield quantification by qPCR in permissive H79 cells (f) and non-permissive human mammary epithelial cells (HMEC) cells (g). Real time PCR reactions were carried out with primers specific to adenovirus 5 hexon region and DNA from viral infected H79, or HMEC cells at quiescence state. Relative viral yield (viral particles/1×10⁵ cells) at different time points was normalized to the input dose determined at 4 hrs post-initial infection;

FIG. 3 are micrographs that show the cytotoxicity to human nonmalignant HMEC cells. HMEC cells (1×10⁵, 6-well plate) were infected with Intemavec, ONYX-411 or wt dl309 (0.1 MOI). The viral cytopathic effect was examined at day 5 post-infection by light microscopy. The proportion of live cells, as defined by cells that lacked cytopathic features, was quantified over two high power (200×) fields;

FIG. 4 is a graph that shows that enhanced tumor growth inhibition of human pancreatic carcinoma H79 xenografts by Internavec. H79 tumor xenografts were induced in athymic nu/nu mice by subcutaneous injection of 3×10⁶ cells. Five daily injections of Internavec (), ONYX-411 (▪) (both at 1×10⁸ pfu) or PBS were given intratumorally when the tumor xenograft reached a size of ≧80 mm³. Data represent mean (±SEM) of tumor xenograft volume (n=5) at different time points post-treatment;

FIG. 5a to 5C summarize the mechanistic characterization of Internavec activity;

FIG. 5a shows the effect of treatment on cell cycle distribution. The frequency distribution of H79 cells following treatment with Internavec (5 MOI) was compared with untreated culture, or following treatment with ONYX-411 or dl309 for the same duration. Cells at different phases (G₀/G₁, S and G₂/M) of cycle were quantified by flow cytometry and ModFit analysis after propidium iodide staining;

FIG. 5b shows the apoptotic activity in Internavec-treated cells. H79 cells were treated with Internavec, ONYX-411 or dl309 (5 MOI), or Fas ligand (0.4 μg/ml) for 72 prior to quantification of 7-AAD incorporation by flow cytometric analysis (see Methods). The level of apoptosis was determined as a function of fluorescence emission by 7-AAD reactive cells at 650 nm. Value represents net increase in % positive cells after background subtraction (untreated culture). Data represent mean+SD of 5-6 separate studies; and

FIG. 5c shows the effect of siRNA^{ras} treatment on gene transcriptional activity. cDNA gene array analysis was carried out with H79 cells treated by Internavec, ONYX-411, or ONYX-411-siRNA^{GFP}. Total RNA were collected at 36 hrs post-treatment. Gene expression signature was determined with the Affymetrix Human Genome U133 Plus 2.0 oligonucleotide probe array chip, and imported into Gene Spring 7.2 software (Silicon Genetics, Santa Clara, Calif.) for differential subtraction for hybridization reactions with a minimum signal intensity of ≧50. Transcripts were compared by hierarchical clustering analysis and identified when they differed from their counterpart in untreated culture by a magnitude of ≧2-fold. This figure represents a heat map of down-regulated gene transcripts that were uniquely downregulated by Internavec and which have previously documented direct relationship with the RAS signaling pathways.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the phrase “nucleic acid molecule” refers to a molecule having nucleotides. The nucleic acid can be single, double, or multiple stranded and can comprise modified or unmodified nucleotides or non-nucleotides or various mixtures and combinations thereof.

As used herein, the term “gene” refers to a nucleic acid that encodes an RNA, for example, nucleic acid sequences including but not limited to structural genes encoding a polypeptide.

As used herein, the term “complementarity” refers to the ability of a nucleic acid to form hydrogen bond(s) with another RNA sequence by either traditional Watson-Crick or other non-traditional types. For use with the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. A percent complementarity indicates the percentage of contiguous residues in a nucleic acid molecule which can form hydrogen bonds (e.g., Watson-Crick base pairing) with a second nucleic acid sequence (e.g., 50%, 60%, 70%, 80%, 90%, and 100% complementary, which is often described as percent homology over the length of a nucleic acid).

As used herein, the terms “aptamer” or “nucleic acid aptamer” refer to a nucleic acid molecule that binds specifically to a target molecule that is distinct from sequence recognized by the target molecule in its natural setting. For example, an aptamer can be a nucleic acid molecule that binds to a target molecule where the target molecule does not naturally bind to a nucleic acid. The target molecule can be any molecule of interest. For example, the aptamer can be used to bind to a ligand binding domain of a protein, thereby preventing interaction of the naturally occurring ligand with the protein.

As used herein, the terms “inhibit” or “down-regulate” refer to the expression of a gene, or level of RNAs or equivalent RNAs encoding one or more protein or subunits of protein complexes such as oncogenes, transcription factors, enzymes, structural proteins, pores, cytokines, receptors, and the like, that is reduced below that observed in the absence of the nucleic acid molecules of the invention. In one embodiment, inhibition or down-regulation with enzymatic nucleic acid molecule is below that level observed in the presence of an enzymatically inactive or attenuated molecule that is able to bind to the same site on the target RNA, but is unable to cleave that RNA. In another embodiment, inhibition or down-regulation with antisense oligonucleotides is below that level observed in the presence of, for example, an oligonucleotide with scrambled sequence or with mismatches. In one embodiment, inhibition or down-regulation of Ras proteins, such as K-Ras, with the nucleic acid molecule is greater in the presence of the siRNA described herein than in its absence.

As used herein, the term “up-regulate” refers to the expression of the gene, or level of RNAs or equivalent RNAs encoding one or more protein or subunits of protein complexes such as oncogenes, transcription factors, enzymes, structural proteins, pores, cytokines, receptors, and the like, is greater than that observed in the absence of the nucleic acid molecules of the invention. In one embodiment, inhibition or down-regulation of Ras proteins, such as K-Ras, with the nucleic acid molecule is less than in the presence of the siRNA described herein than in its absence.

As used herein, the term “modulate” refers to the expression of the gene, or level of RNAs or equivalent RNAs encoding encoding one or more protein or subunits of protein complexes such as oncogenes, transcription factors, enzymes, structural proteins, pores, cytokines, receptors, and the like, that is up-regulated or down-regulated, such that the expression, level, or activity is greater than or less than that observed in the absence of the siRNA molecules of the invention.

As used herein, the phrase “enzymatic nucleic acid molecule” refers to a nucleic acid molecule that has complementarity in a substrate binding region to a specified gene target, and also has an enzymatic activity which is active to specifically cleave target RNA.

As used herein, the term “siRNA” refers to nucleic acids such DNA or RNA that is in its native form or that can be modified at the base, sugar, and/or phosphate groups. When the siRNA is an enzymatic nucleic acid it may be a ribozyme, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme, DNA enzyme or a catalytically active regulatable ribozyme. The specific siRNA molecules described herein do not limit the invention and those skilled in the art will recognize that all that is important in an enzymatic nucleic acid molecule of this invention is that it has a specific substrate binding site that is complementary to one or more of the target nucleic acid regions, and that it have nucleotide sequences within or surrounding that substrate binding site which impart a nucleic acid cleaving and/or ligation activity to the molecule.

The siRNA may target one or more gene targets, which are genes that encodes a protein selected from, e.g., amyloid protein, amyloid precursor protein, angiostatin, endostatin, METH-1, METH-2, Factor IX, Factor VIII, collagen, cyclin dependent kinase, cyclin D1, cyclin E, WAF 1, cdk4 inhibitor, MTS1, cystic fibrosis transmembrane conductance regulator, IL-1, IL-2, IL-3, IL-4, IL-5, IL-6, IL-7, IL-8, IL-9, IL-10, IL-11, IL-12, IL-13, IL-14, IL-15, IL-16, IL-17, erythropoietin, G-CSF, GM-CSF, M-CSF, SCF, thrombopoietin, BNDF, BMP, GGRP, EGF, FGF, GDNF, GGF, HGF, IGF-1, IGF-2, KGF, myotrophin, NGF, OSM, PDGF, somatotrophin, TGF-β, TGF-α, VEGF, interferon, TNF-.alpha., TNF-.beta., cathepsin K, cytochrome p-450, farnesyl transferase, glutathione-s transferase, heparanase, HMG CoA synthetase, n-acetyltransferase, phenylalanine hydroxylase, phosphodiesterase, ras carboxyl-terminal protease, telomerase, TNF converting enzyme, E-cadherin, N-cadherin, selectin, CD40, 5-α reductase, atrial natriuretic factor, calcitonin, corticotrophin releasing factor, glucagon, gonadotropin, gonadotropin releasing hormone, growth hormone, growth hormone releasing factor, somatotropin, insulin, leptin, luteinizing hormone, luteinizing hormone releasing hormone, parathyroid hormone, thyroid hormone, thyroid stimulating hormone, immunoglobulin, CTLA4, hemagglutinin, major histocompatibility factor (MHC), VLA-4, kallikrein-kininogen-kinin system, CD4, sis, hst, ras, abl, mos, myc, fos, jun, H-ras, ki-ras, c-fms, bcl-2, L-myc, c-myc, gip, gsp, HER-2, bombesin receptor, estrogen receptor, GABA receptor, EGFR, PDGFR, FGFR, NGFR, GTP-binding regulatory proteins, interleukin receptors, ion channel receptors, leukotriene receptor antagonists, lipoprotein receptors, opioid pain receptors, substance P receptors, retinoic acid and retinoid receptors, steroid receptors, T-cell receptors, thyroid hormone receptors, TNF receptors, tissue plasminogen activator; transmembrane receptors, calcium pump, proton pump, Na/Ca exchanger, MRP 1, MRP2, P170, LRP, cMOAT, transferrin, APC, brca1, brca2, DCC, MCC, MTS1, NF1, NF2, nm23, p53 and Rb.

As used herein, the term “homology” refers to the nucleotide sequence of two or more nucleic acid molecules that are partially or completely identical. As used herein, the phrase “antisense nucleic acid” refers to a non-enzymatic nucleic acid molecule that binds to target RNA by means of RNA--RNA or RNA-DNA or RNA-PNA (protein nucleic acid) interactions and alters the activity of the target RNA as is well known to the skilled artisan. Typically, antisense molecules are complementary to a target sequence along a single contiguous sequence of the antisense molecule. In certain embodiments, an antisense molecule binds to substrate such that the substrate molecule forms a loop, and/or an antisense molecule can bind such that the antisense molecule forms a loop. Antisense DNA can be used to target RNA via DNA-RNA interactions, thereby activating RNase H, which digests the target RNA in the duplex. The antisense oligonucleotides can include one or more RNase H activating regions that are capable of activating RNAse H cleavage of a target RNA.

As used herein, the terms “double stranded RNA” or “dsRNA” refer to a double stranded RNA that matches a predetermined gene sequence or target that is capable of activating cellular enzymes that degrade the corresponding messenger RNA transcripts of the gene. Also referred to as small interfering RNA (siRNA), these inhibit gene expression. As used herein, the term “double stranded RNA” or “dsRNA” refers to a double stranded RNA molecule capable of RNA interference “RNAi”, including short interfering RNA “siRNA” as taught in International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914, relevant portions incorporated herein by reference.

The term “gene silencing” as defined herein is used to describe the phenomenon of reduced or repressed translation of mRNA into a protein. Examples of aptamer-mediated “gene silencing” include short ssDNA, ssRNA or dsRNA, that may vary from 15 to 70 nt long (for precursors) that repress protein expression by specific or non-specific degradation of mRNA and/or binding to the mRNA in a location, time and manner that inhibits the cellular translational complex from translating the mRNA into protein. Degradation may occur, e.g., by non-specific antisense DNA/RNA duplex formation and resulting RNase H-type RNA degradation or sequence specific DICER/RISC mediated mRNA degradation. The term “RNA interference” (RNAi) is defined herein as gene silencing by cleavage of perfectly complementary mRNA, which in mammals is mediated by 21-23 nt small, interfering RNAs (siRNAs) which are double-stranded, and which are produced by Dicer cleavage of long ds RNA, with the resulting siRNA incorporated into an RNA-induced silencing complex (RISC). As used herein, the term “gene silencing” also applies to miRNA repression of translation, in which the miRNA complementarity is imperfect but the nucleic acids are able to repress (lower or eliminate) gene translation.

As used herein, the term a “target gene” refers to a gene derived from the cell, a transgene (e.g., a gene construct inserted at an ectopic site in the genome of the cell), or a gene from a pathogen that is capable of infecting an organism from which the target cell is derived. Depending on the particular target gene and the dose of siRNA delivered, this process may provide partial or complete loss of function for the target gene. In some cases, gene silencing of a target gene may be a reduction or loss of gene expression in at least 99% of targeted cells. Generally, gene silencing may be shown by the inhibition of gene expression such that the level of protein and/or mRNA product from a target gene in a cell is absent or reduced about 5, 10, 20, 30, 50, 75 80, 90 or even about 100% (i.e., an observable decrease within the limits of detection of the assay selected to measure gene silencing). Specificity of the siRNA refers to the ability of the siRNA to inhibit the target gene without manifest effects on other genes of the cell. The consequences of inhibition may be confirmed by examination of phenotypic changes (i.e., outward properties of the cell or organism) or by genotypic or biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For siRNA-mediated inhibition in a cell line or whole organism, gene expression may be assayed by use of a reporter or drug resistance gene whose protein product is easily assayed. Reporter genes may include, e.g., acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Furthermore, the detection of gene silencing may even be by using multiple selectable markers are available that confer resistance to ampicillin, bleomycin, chloramphenicol, gentamycin, hygromycin, kanamycin, lincomycin, methotrexate, phosphinothricin, puromycin, and tetracycline.

Depending on the assay used to measure gene silencing using the siRNA of the present invention, quantitation of the amount of gene expression allows one to determine a degree of inhibition which is greater than about 5%, 10%, 20%, 25%, 33%, 50%, 60%, 75%, 80%, 90%, 95% or about 99% as compared to a target cell that has not been not treated according to the methods of the present invention. The siRNA disclosed herein may permit the use of lower doses of injected material and longer times after administration of, e.g., dsRNA aptamers resulting in the inhibition of a smaller fraction of cells (e.g., at least about 10%, 20%, 50%, 75%, 90%, or about 95% of targeted cells). Quantitation of gene expression in a cell may show similar amounts of silencing that depends on the level of accumulation of target mRNA and/or translation of target protein. For example, the efficiency of inhibition may be determined by assessing the amount of gene product in the cell: mRNA may be detected with a hybridization probe having a nucleotide sequence outside the region used for the inhibitory double-stranded RNA, or translated polypeptide may be detected with an antibody raised against the polypeptide sequence of that region.

As used herein, the term “vector” is used in reference to nucleic acid molecules that transfer DNA segment(s) from one cell to another. The vector may be further defined as a “replication-competent, oncolytic adenovirus” that includes promoters that permit expression or transcription of a nucleic acid segment that has been introduced into the vector that includes a promoter operatively linked to the siRNA sequence, or one designed to cause such a promoter to be introduced. The vector may exist in a state independent of the host cell chromosome, or may be integrated into the host cell chromosome.

As used herein, the term “oncolytic viruses” is used to describe viruses that infect and replicate in cancer cells, killing the cancer cells and leaving normal cells largely unaffected. The replication-competent oncolytic viruses replicate in cancer cells, e.g., human cancer cells in a patient, and also transcribe one or more genes under control of the viral or other promoters. The present invention takes advantage of the oncolytic viruses to deliver both oncolysis but also the delivery of critical siRNAs that are able to target specific genes within the cancer cell that may otherwise increase the resistance of the cancer cell to oncolysis.

The term “host cell” refers to cells that have been engineered to contain nucleic acid segments or a replication-competent, oncolytic adenovirus that includes one or more siRNA genes, or altered segments, whether archeal, prokaryotic, or eukaryotic. Thus, engineered, or recombinant cells, are distinguishable from naturally occurring cells that do not include recombinantly introduced genes through the hand of man.

For use in a human patient, the siRNA delivering vector of the present invention will generally be prepared in a pharmaceutically acceptable form. The oncolytic virus siRNA vector of the present invention may be delivered as a pharmaceutically acceptable salt thereof is administered to a patient, e.g., a mammal, a human or other, suffering from a disease whose progression is associated with a target RNA-ligand interaction in vivo. The oncolytic virus siRNA vector is administered to a patient for treating or preventing a disease.

As used herein, “treatment” or “treating” refers to an amelioration of a disease, or at least one discernible symptom thereof. In another embodiment, “treatment” or “treating” refers to an amelioration of at least one measurable physical parameter, not necessarily discernible by the patient. In yet another embodiment, “treatment” or “treating” refers to inhibiting the progression of a disease, either physically, e.g., stabilization of a discernible symptom, physiologically, e.g., stabilization of a physical parameter, or both. In yet another embodiment, “treatment” or “treating” refers to delaying the onset of a disease.

In certain embodiments, the oncolytic virus siRNA vector or a pharmaceutically acceptable salt thereof is administered to a patient as a preventative measure against a disease associated with an RNA-ligand interaction in vivo. As used herein, “prevention” or “preventing” refers to a reduction of the risk of acquiring a disease. In one embodiment, the oncolytic virus siRNA vector or a pharmaceutically acceptable salt thereof is administered as a preventative measure to a patient. The patient may have a genetic predisposition to a disease, such as a family history of the disease, or a non-genetic predisposition to the disease. Accordingly, the oncolytic virus siRNA vector can be used for the treatment of one manifestation of a disease and prevention of another.

When administered to a patient, the oncolytic virus siRNA vector or a pharmaceutically acceptable salt thereof is preferably administered as component of a composition that optionally comprises a pharmaceutically acceptable vehicle. The composition can be administered orally, or by any other convenient route, for example, by infusion or bolus injection, by absorption through epithelial or mucocutaneous linings (e.g., oral mucosa, rectal, and intestinal mucosa, etc.) and may be administered together with another biologically active agent. Administration can be systemic or local. Various delivery systems are known, e.g., encapsulation in liposomes, microparticles, microcapsules, capsules, etc., and can be used to administer the oncolytic virus siRNA vector and pharmaceutically acceptable salts thereof.

Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The mode of administration is left to the discretion of the practitioner. In some instances, administration will result in the release of the oncolytic virus siRNA vector into the bloodstream.

In specific embodiments, it may be desirable to administer the oncolytic virus siRNA vector locally, which may be achieved, for example, and not by way of limitation, by local infusion during surgery, topical application, e.g., in conjunction with a wound dressing after surgery, by injection, a catheter, a suppository, or an implant (e.g., a porous, non-porous, gelatinous, membranes or fibers).

In certain embodiments, the oncolytic virus siRNA vector is delivered into the central nervous system by any suitable route, including intraventricular, intrathecal and epidural injection. Intraventricular injection may be facilitated by an intraventricular catheter, for example, attached to a reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the oncolytic virus siRNA vector and pharmaceutically acceptable salts thereof can be formulated as a suppository, with traditional binders and vehicles such as triglycerides. In another embodiment, the oncolytic virus siRNA vector and pharmaceutically acceptable salts thereof can be delivered in a vesicle, in particular a liposome.

In yet another embodiment, the oncolytic virus siRNA vector and pharmaceutically acceptable salts thereof can be delivered in a controlled release system, a pump, a polymeric material, a controlled-release system and the like. Generally, the oncolytic virus siRNA vector can be placed in proximity of the target cell, thus requiring only a fraction of the systemic dose.

Compositions that include the oncolytic virus siRNA vector or a pharmaceutically acceptable salt thereof (“test compound compositions”) can additionally have a suitable amount of a pharmaceutically acceptable vehicle so as to provide the form for proper administration to the patient.

As used herein, the term “pharmaceutically acceptable” refers to approval by a regulatory agency of the Federal or a state government or listed in the U.S. Pharmacopeia or other generally recognized pharmacopeia for use in animals, mammals, and more particularly in humans.

As used herein, the term “vehicle” refers to a diluent, adjuvant, excipient, or carrier with which a compound of the invention is administered. Examples of pharmaceutical vehicles include liquids (water, isotonic solutions, saline and the like) and oils (e.g., petroleum, animal, vegetable or synthetic origin, such as peanut oil, soybean oil, mineral oil, sesame oil and the like). The pharmaceutical vehicles may be saline and/or include one or more thickeners such as gum acacia, gelatin, starch paste, talc, keratin, colloidal silica, urea, and the like. In addition, auxiliary, stabilizing, thickening, lubricating and coloring agents may be used that are pharmaceutically acceptable and/or that help stabilize the oncolytic virus siRNA vector of the present invention. When administered to a patient, the pharmaceutically acceptable vehicles are sterile. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Test compound compositions, if desired, can also contain minor amounts of wetting or emulsifying agents, or pH buffering agents.

The oncolytic virus siRNA vector may be provided and/or stored in solutions, suspensions, emulsion, tablets, pills, pellets, dry capsules, capsules containing liquids, powders, sustained-release formulations, suppositories, emulsions, aerosols, sprays, suspensions, or any other form suitable for use. Examples of suitable pharmaceutical vehicles are described in Remington's Pharmaceutical Sciences, Alfonso R. Gennaro ed., Mack Publishing Co. Easton, Pa., 19th ed., 1995, relevant portions incorporated herein by reference.

The oncolytic virus siRNA vector or a pharmaceutically acceptable salt thereof is formulated in accordance with routine procedures as a pharmaceutical composition adapted for oral administration to human beings. Compositions for oral delivery may be in the form of tablets, lozenges, aqueous or oily suspensions, granules, powders, emulsions, capsules, syrups, or elixirs, for example. Orally administered compositions may contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation.

When in tablet or pill form, the compositions can be coated to delay disintegration and absorption in the gastrointestinal tract thereby providing a sustained action over an extended period of time. Selectively permeable membranes surrounding an osmotically active driving compound are also suitable for orally administered compositions. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, and the like. Such vehicles are preferably of pharmaceutical grade. Typically, compositions for intravenous administration comprise sterile isotonic aqueous buffer and/or a solubilizing agent.

The oncolytic virus siRNA vector or a pharmaceutically acceptable salt thereof can be formulated for intravenous or intra-arterial administration. Compositions for intravenous administration may optionally include a local anesthetic such as lidocaine to lessen pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent.

When the oncolytic virus siRNA vector or a pharmaceutically acceptable salt thereof is to be administered by infusion, it can be dispensed, for example, with an infusion bottle containing sterile pharmaceutical grade water or saline. Where the oncolytic virus siRNA vector or a pharmaceutically acceptable salt thereof is administered by injection, an ampoule of sterile water for injection or saline can be provided so that the ingredients may be mixed prior to administration.

The amount of the oncolytic virus siRNA vector or a pharmaceutically acceptable salt thereof that will be effective in the treatment of a particular disease will depend on the nature of the disease, and can be determined by standard clinical techniques. In addition, in vitro or in vivo assays may optionally be employed to help identify optimal dosage ranges. The precise dose to be employed will also depend on the route of administration, and the seriousness of the disease, and should be decided according to the judgment of the practitioner and each patient's circumstances. Suitable dosage ranges for oral administration may be determined, as defined by plaque forming units (pfu) of the oncolytic virus siRNA vector or a pharmaceutically acceptable salt thereof per kilogram body weight per day as will be known to those of skill in the art.

Suitable biotherapeutic dosage ranges for intravenous (i.v.) administration are about 0.01 milligram to about 100 milligrams per kilogram body weight per day, generally about 1×10⁷ to about 1×10⁸ plaque forming units (pfu) per kilogram body weight per day. Suitable dosage ranges for intranasal administration or suppository dosage will be determined for a compound of the invention per kilogram body weight per day and comprise active ingredient in the range of about 0.5% to about 10% by weight.

Recommended dosages for intradermal, intramuscular, intraperitoneal, subcutaneous, epidural, sublingual, intracerebral, intravaginal, transdermal administration or administration by inhalation are in the range of about 10⁷ to about 10⁹ plaque forming units (pfu) per kilogram of body weight per day. Suitable doses for topical administration are in the range of about 0.001 milligram to about 1 milligram, depending on the area of administration. Effective doses may be extrapolated from dose-response curves derived from in vitro or animal model test systems. Such animal models and systems are well known in the art.

The oncolytic virus siRNA vector and pharmaceutically acceptable salts thereof are preferably assayed in vitro and in vivo, for the desired therapeutic or prophylactic activity, prior to use in humans. For example, in vitro assays can be used to determine whether it is best to administer the oncolytic virus siRNA vector, a pharmaceutically acceptable salt thereof, and/or another therapeutic agent. Animal model systems can be used to demonstrate safety and efficacy.

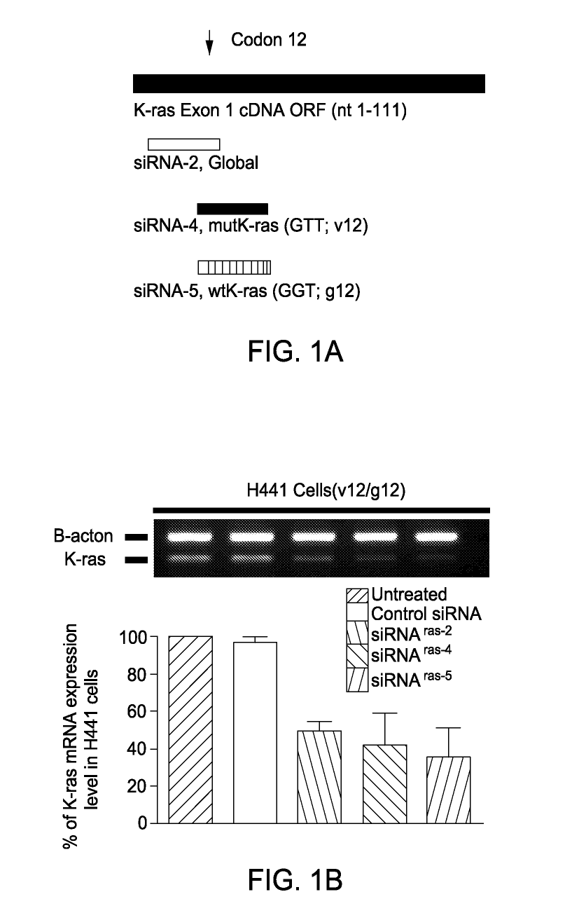

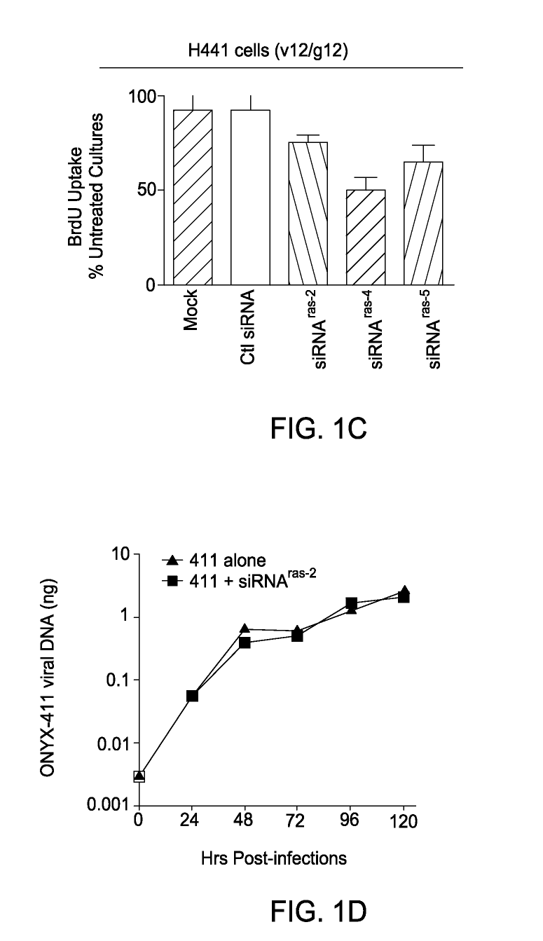

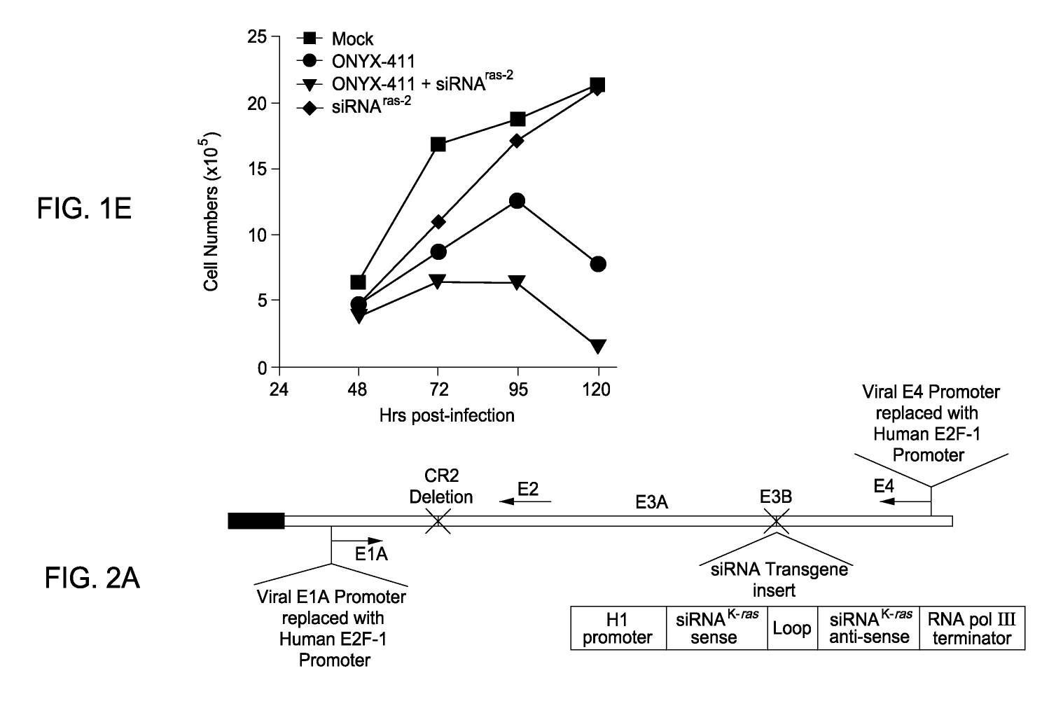

To characterize the interaction of siRNA-mediated oncogene knockdown and viral oncolysis, we elected the use of siRNAs against the K-ras proto-oncogene (siRNA^{ras}), based on our prior success of inducing tumor growth inhibition by ribozyme-mediated K-ras knockdown (18). A number of siRNA^{ras}s were designed against “stepped” nucleotide sequences at exon 1, codons 8-14 (FIG. 1a), one of the three “hotspots” of ras oncogene mutations and a region previously demonstrated to be susceptible to siRNA knockdown (7,8). Prediction of a lack of “off-target” effects against irrelevant gene sequences was determined by BLAST search of the NCBI GeneBank database. Coinfection studies were carried out with ONYX-411, a late generation, conditional replicative, oncolytic adenovirus, whose anti-tumor potency was 10-100-fold higher than ONYX-015 (19, 20). A549 human lung cancer cells were used as target, in view of their permissiveness to adenoviral infection and moderate resistance to viral oncolysis at low multiplicities of infection (MOIs) (19). To simulate peak siRNA transgene expression at the “late” phase of viral replication, siRNA^{ras-2} with demonstrated K-ras knockdown (FIG. 1b-1c) was introduced by lipofection at 24 hrs post-viral infection. siRNA^{ras-2} targets the consensus K-ras exon 1 sequence upstream of codon 12 (FIG. 1a) and hence is applicable for targeting A549 cells with a K-ras^{s12/s12} genotype. siRNA^{ras-2} alone produced the highest level of growth inhibitory response at 48 hrs post-treatment, when viable tumor cells were reduced by 35%. By comparison, low dose ONYX-411 (MOI of 5) reduced viable tumor cells by 49-63% at 72-120 hrs post-viral infection (FIG. 1e). Co-treatment with siRNA^{ras-2} and ONYX-411 displayed an additive anti-tumor effect, resulting in a >90% reduction of viable tumor cells at 120 hrs post-viral infection. These findings indicate that the antitumor effect was augmented and prolonged through siRNA-mediated ras knockdown and viral oncolysis. siRNA^{ras} cotreatment did not affect viral yield of ONYX-411-infected tumor cells, according to real time PCR quantification (FIG. 1d).

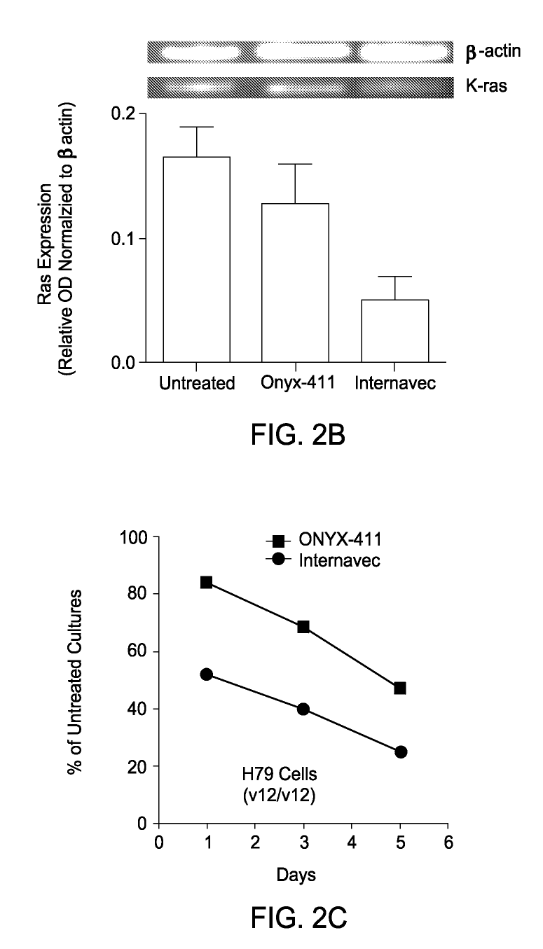

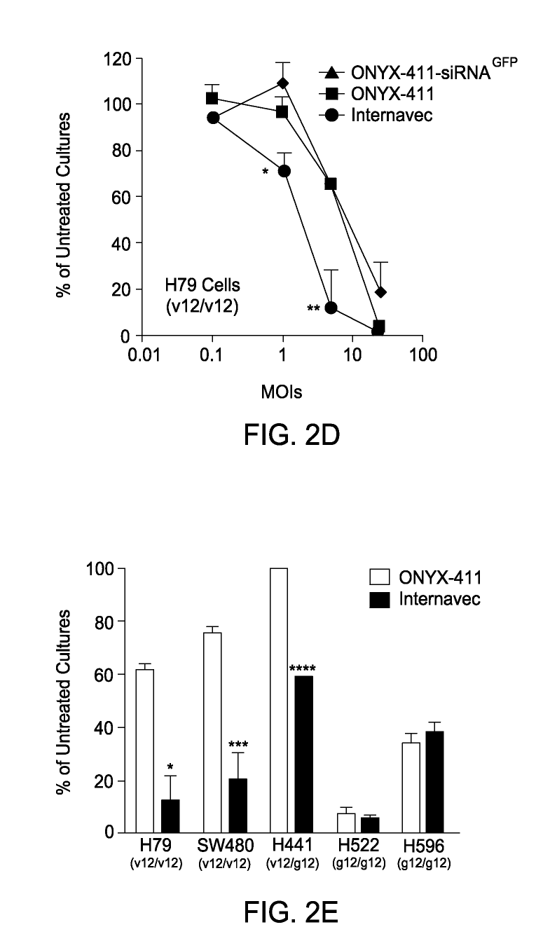

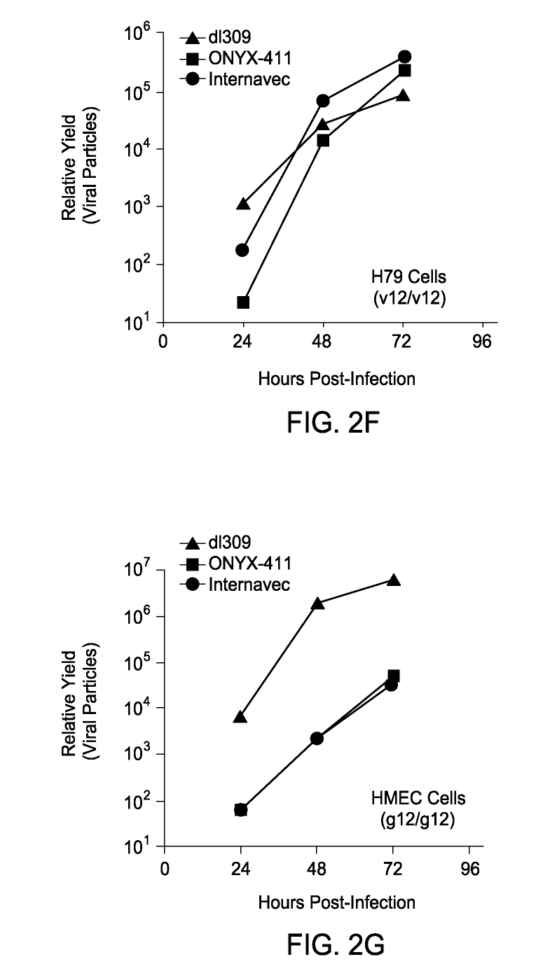

Based on these favorable findings, an siRNA was cloned that specifically targets the common K-ras oncogene mutant (K-ras^{v12}) into the E3B region of ONYX-411. siRNA^{ras-4}, chosen for its highly effective tumor growth-inhibitory activity during siRNA screening analyses (FIG. 1c), was integrated in a short hairpin (sh) configuration encompassing a single Pol III (H1) promoter upstream of tandem siRNA sense and antisense encoding sequences that are separated by a 3-9 nucleotide intervening loop (FIG. 2a). The Pol III promoter causes termination after the second uridine and produces a transcript that mimics the natural siRNA configuration after Dicer processing. A similarly configured shRNA transgene for the reported gene luciferase was recently shown to be highly effective when delivered from the E4 region of the replication competent adenovirus AdΔ24E3 (22). The E3B region was used as the cloning site, in view of prior findings that genes inserted in this region were coordinately expressed as part of the “major late transcription unit” following initiation of viral DNA replication (20,21), hence limiting transgene expression and activity to host cells that are permissive to viral replication.

Molecular integrity of siRNA^{ras-4} in the ONYX-411-siRNA construct, named Internavec, was confirmed by PCR and bi-directional DNA sequence. Functional evaluations were carried out with the homozygous K-ras^{v12} mutant Capan-1 (H79) pancreatic cancer cells. Internavec treatment, but not the parental ONYX-411 virus, significantly reduced K-ras mRNA expression at 48 hrs post-treatment (63% reduction as compared with untreated control, p<0.02; student's t test; FIG. 2b). Viable H79 cells were reduced by 48%, 61% and 75% on days 1, 3, and 5 post-Internavec treatment according to trypan blue exclusion analyses, as compared with 16%, 31% and 53%, respectively in ONYX-411-treated cultures (FIG. 2c). MTT analysis confirmed that cellular activity was reduced by 88% in Internavec-treated H79 cells (5 pfu) at day 5, as compared with a 39% reduction by parental ONYX-411 or the control construct carrying the siRNA^{GFP} (FIG. 2d).

Comparative analysis indicates that the effective dose for inhibiting cancer cell growth by 50% (ED₅₀) was reduced by approximately 10-fold through incorporation of the siRNA^{ras-4} transgene (MOI of 1.03 as compared with an MOI of 11.6 by parental ONYX 411 and MOI of 14.7 by ONYX-411-siRNA^{GFP}; FIG. 2d). K-ras mutations in human cancer are localized in a limited number of “hotspots” (codon 12, 13, or 61) that differed from wt by a single nucleotide substitution (18). The enhanced antitumor activity of Internavec was evident in human cancer lines expressing the relevant, homozygous K-ras^{v12} mutation (SW480 colon carcinoma), as well as in H441 lung cancer cells that have a heterozygous K-ras^{v12/wt} phenotype and were resistant to ONYX-411 oncolysis (FIG. 2e). By contrast, incorporation of siRNA^{ras} did not markedly improve anti-tumor activity in cancer lines lacking the relevant K-ras mutation (H596, H522, both with wt K-ras^{g12/g12} phenotypes) (FIG. 2e). These findings showed that Internavec retains its specific knockdown activity of K-ras^{v12}, which translates into an additive anti-tumor response with ONYX-411 oncolytic activity.

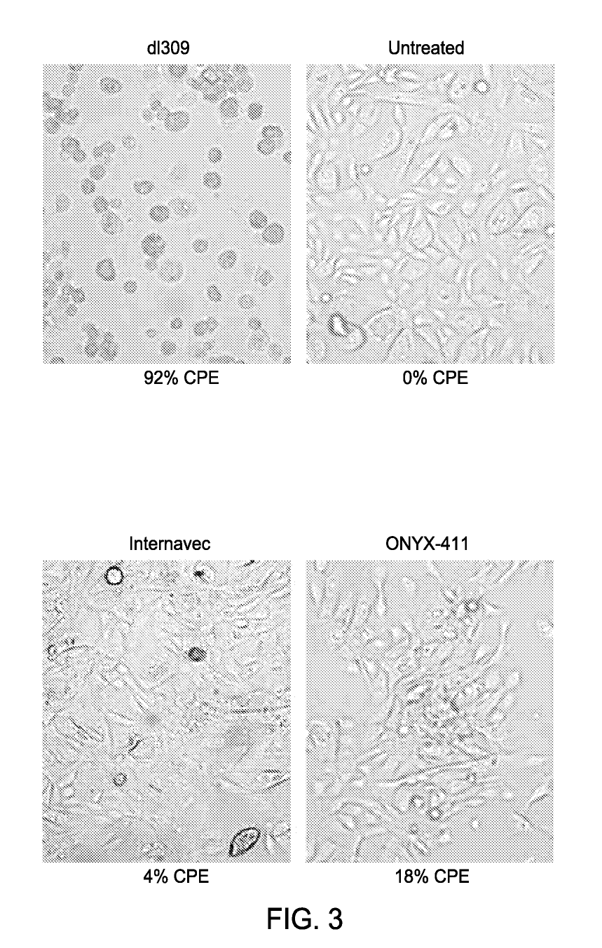

ONYX-411 carries an EIA deletion and E2F-1 conditional promoters in the E1 and E4 regions, hence limiting replication to cancer cells with a defective retinoblastoma tumor suppressor protein (pRb) pathway and E2F-1 overexpression (20,21). We examined the cytopathic effects of Internavec as compared with ONYX-411 on nonmalignant, human epithelial HMEC cells, in order to determine the impact of siRNA arming on the conditional replicative activity of ONYX-411. Both ONYX-411 and Internavec produced low levels of toxicity (loss of viability in 18% and 4% of total cells, respectively), as compared with cytopathic effects that were observed in >90% of HMEC cells following wt, dl309 viral infection (FIG. 3). Internavec and ONYX-411 displayed comparable replicative activities in permissive H79 cancer cells (FIG. 2f), whereas both viruses were attenuated by approximately 750-fold in HMEC cells as compared with dl309 (FIG. 2g). These findings indicate that integration of siRNA^{ras} did not produce added cytotoxicity in ONYX-411 non-permissive HMEC cells.

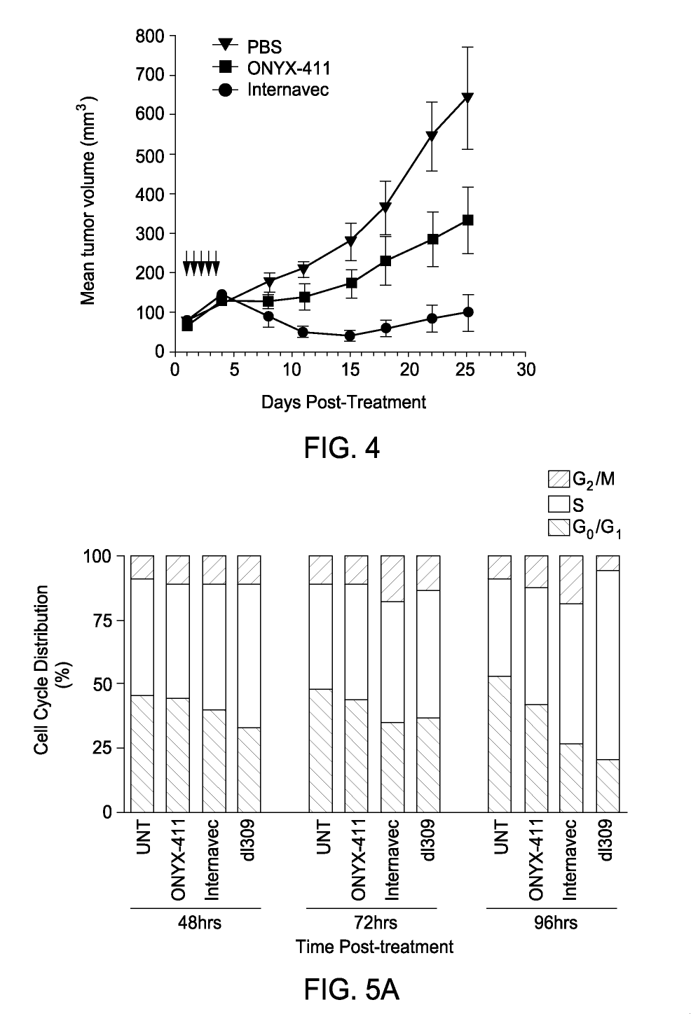

The in vivo antitumor efficacy of Internavec was examined with a subcutaneous H79 xenograft model in athymic nu/nu mice. Internavec at 1×10⁸ pfu completely inhibited tumor-growth in 3 of 5 treated mice for up to 32 days post-treatment. The mean reduction of 85.5% in all 5 Internavec-treated tumors was significantly more effective as compared with the same treatment dose of ONYX-411 (47.8% growth reduction, p=0.03), or ONYX-411-siRNA^{GFP} (44.1%, p=0.03) (FIG. 4). These findings showed that integration of siRNA^{ras} knockdown activity markedly enhanced viral oncolysis by ONYX-411 in vivo.

Currently, the precise mechanism by which a conditional replicative, adenovirus effects tumor cell kill is unclear. The self-limiting disease course of wild type adenoviral infections in immunocompromised patients is indicative of limited virulence (14). Host cells with viral protein expression are seen commonly post-infection, indicative of an aborted viral replicative process and/or a latent infection state (14, 22). Coupled with the common defects in apoptotic and interferon-induction pathways in cancer cells, the aborted oncolytic process is an explanation for incomplete clinical responses seen in oncolytic virotherapy trials (15, 17). The incorporation of a regulatory, small RNA transgene that can disrupt cell survival mechanisms, such as through K-ras knockdown, would conceivably tilt the cellular balance towards cancer cell death.

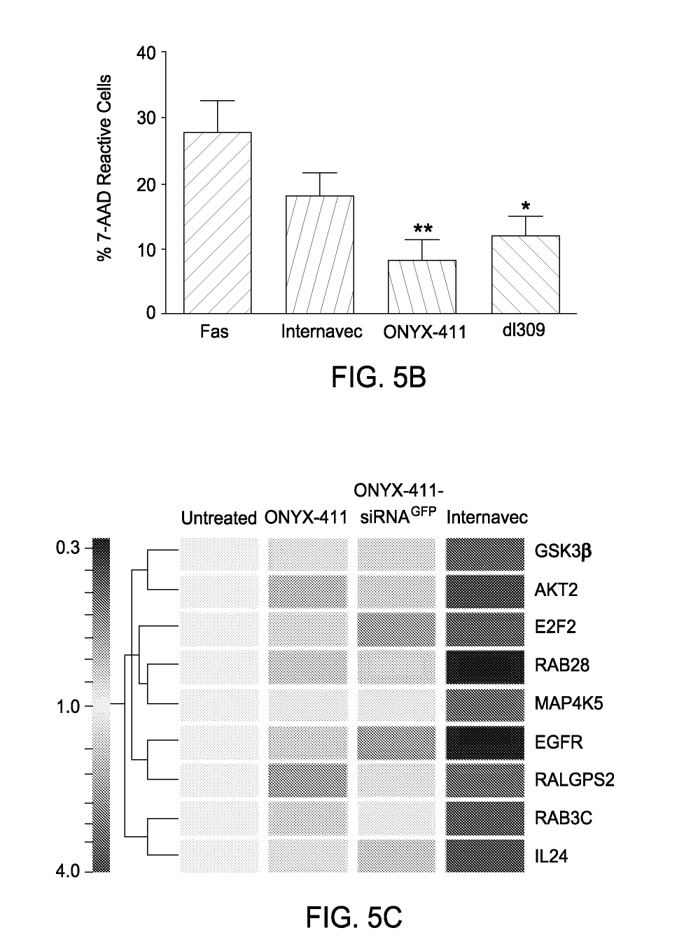

Internavec treated cultures displayed comparable, albeit moderately elevated viral replicative activity as ONYX-411 treatment in permissive H79 cells (FIG. 2f), suggesting that facilitation of viral replication may not be the primary contribution for enhanced tumor cell kill. Cell cycle analyses were carried out to characterize the treatment impact on the growth fraction (S+G₂/M) distribution of H79 cells (FIG. 3a). For untreated cultures, the % cycling cells remained relatively constant over 96 hours (54-47%), as compared with progressively increasing growth fractions in virus treated cultures. As shown in a representative study (FIG. 5a), S+G₂/M cycling cells constituted 80% of dl309-treated cells, 73% of Internavec-treated cells, and 58% of ONYX-411 treated cells at 96 h post-infection. These findings were consistent with previous reports (24, 25) of early adenoviral genes overriding the fundamental control of the host cell cycle, forcing progression into S phase. Compared with ONYX-411, Internavec appeared more effective in driving cells into growth phase at the level that approached that of dl309 (FIG. 5a). Increases in S phase (55%) and G₂/M (18%) compartments were seen, as compared with ONYX-411 (46% and 12%, respectively). Cell cycle blockage was accompanied by modestly increased apoptotic activity by Internvec treatment (18+3.5% as compared 8.2+3.3% by ONYX-411; p<0.001) (FIG. 5b). An activated K-RAS has been reported to modulate cycling cells through a wide spectrum of cell cycle regulators (26), but in particular facilitates transition from G₂/M into the next cycle (27). The findings with Internavec were consistent with cell cycle blockage events that resulted from early viral gene expression as well as K-ras knockdown, culminating in S and G₂/M phase arrests. Previously reported antiapoptotic activities of adenoviral E1B and E3 genes (14,15) may account for the observed low levels of apoptosis. Hence enhanced tumor cell kill by Internavec may engage alternative pathways of cell death induction (28).

To define the transcriptional impact of Internavec treatment, gene array analysis was carried out with the Affymetrix Human Genome U133 Plus 2.0 oligonucleotide probe array. Hybridization reactions were normalized to that by RNA from untreated cultures. Differential subtraction of the gene profile signature of ONYX-411-treated cultures from Internavec-treated cells yielded 198 genes that were uniquely downregulated by Internavec (≧2 fold). Using Affymetrix and David 2.0 software, 41 genes were assigned to cell cycle, cell-cell interaction and signal transduction, RNA and protein metabolism and stress and immune response (Table 1). In particular, 4 were associated with RAS-dependent modulation of cell cycle and proliferation whose downregulation may be attributable to upstream ras PTGS, namely protein kinase β (AKT2), glycogen synthase kinase-3 β (GSK3β), the transcription factor E2F2, and the mitogen-activated protein kinases MAP4K5 (FIG. 5c and Table 1). AKT2 is the only member of the Akt/PKB family implicated in several types of human malignancies, and known to be collaterally activated by RAS (28). Akt2 knockdown has been shown to reduce viable cell numbers, suppresses tumor clonogenicity, cell migration and invasion, and increases apoptosis and necrosis (29), and likely contributes to the antitumor outcome of Internavec treatment. The impact of Internavec treatment on protein translation is currently being investigated by proteomic analysis.

Recently, Andersson and coworkers (30) found that expression of the adenoviral genes, virus-associated (VA) RNAI and VA RNAII, led to suppression RNA interference activity, possibly through competitively interfering with Dicer or RISC activity. The validity of this phenomenon needs to be further confirmed, since the studies were performed only in HEK293 cells with integrated viral DNA and corresponding helper activities for viral gene expression and replication. RNAi suppression was evident only when high concentrations (up to 108 copies/cell) of VA RNA were present at a very late phase of the replicative cycle (29). By comparison, the study showed a maximal siRNA-additive effect at the earlier time frame of 48-72 hrs post-infection. Other siRNAs have been integrated into the E3 region of non-replicative adenoviral constructs with a wild type VA RNAI and RNAII configuration. Their RNAi activity was also unaffected by the wild type VA RNAI and RNAII phenotype of the vector backbone [Rao D, personal communications]. Hence further studies are needed with viral permissive cancer lines to better define the interaction of VA RNA expression and RNAi in human cancer cells.

Therefore, the present inventors designed, constructed and used for the first time an oncolytic viral-transgene construct to attain sequence specific PTGS within the tumor microenvironment. These findings indicate that siRNA-mediated K-ras knockdown manifested as enhanced cell cycle blockage via multiple molecular pathway perturbations, and led to an additive antitumor response with viral oncolysis.

siRNAs and DNA oligonucleotides. K-ras siRNA gene target sequence were designed targeting the K-ras exon 1 around commonly mutated codon 12, and lacks homology to other known human genes as examined by BLAST search of the NCBI GeneBank database. siRNA^{ras-2} targets a consensus c-K-ras sequence upstream of codon 12, whereas siRNA^{ras}-4 and -5 are specific for the ras^{v12} and ras^{wt} (g12) sequence, respectively (FIG. 1a). Customized siRNA^{ras} RNA duplex as well as pre-defined control, non-silencing siRNA duplex at HPP grade (Qiagen, Valencia, Calif.) were introduced into target cells by lipofection (GeneSilencer siRNA transfection reagent, Gene Therapy Systems, Inc., San Diego, Calif.) according to manufacturer's instructions. For the cloning of siRNA transgene, DNA oligonucleotides for siRNA^{ras-4} and control siRNA^{GFP} (pre-defined sequence from Ambion, Austin, Tex.) were synthesized by Integrated DNA technologies. All other DNA oligos used, including for RT-PCR, qPCR, PCR and DNA sequencing, were synthesized by Sigma-Genesys.

Cell lines. The human lines H441, A549, H522, H596 (all nonsmall cell lung cancers), H79 (pancreatic carcinoma), SW480 (colon carcinoma), and HEK293 (embryonic kidney) were obtained from American Type Culture Collection (ATCC, Manassas, Va.). H441, H522, and H596 cells were cultured in 10% fetal bovine serum (FBS, Atlanta Biological, Atlanta, Ga.)+RPMI 1640 (ATCC), A549 cells in 10% FBS+Dulbecco's Minimal Eagle's medium (DMEM, Invitrogen), H79 cells in 20% FBS+Iscove's Modified Dulbecco's Medium (IMDM, ATCC); 293 cells in 10% heat-inactivated horse serum+DMEM+1:100 diluted antibiotics-antimycotics (penicillin 10,000 U/mL; streptomycin, 10,000 μg/mL; and amphotericin B, 25 μg/mL) (Invitrogen, Carlsbad, Calif.). Normal human mammary epithelial cells (HMEC) (Cambrex, Walkersville, Md.) were initially cultured in T25 flask for 1-2 passages and then seeded on 24-well plates at 1×10⁵ cells/well in 1.0 ml of MEGM Bullet kit medium (Cambrex) and renewed with fresh media every 3 days. The culture is allowed to reach confluence, then extended for an additional 10-14 days in order to induce growth quiescence prior to viral infection (20).

Generation of Internavec. The cDNA sequence of siRNA^{ras-4} was cloned into the ONYX-411 E3B region (ONYX Pharmaceuticals). Following restriction enzyme digestion, the hairpin siRNA template [forward strand: 5′-GGATCC+siRNA^{ras-4} sense strand+loop (TTCAAGAGA)+siRNA^{ras-4} antisense strand+polymerase III terminator (TTTTTT)+GGAAA was ligated into the pSilencer H1 plasmid (Ambion, Austin, Tex.). The H1 promoter-K-ras siRNA PCR product containing ClaI/SwaI restriction sites was generated by PCR, using the specific primers (forward primers: 5′-AGGCTAATCGATCATATTTGCATGTCGCTATGTG (SEQ ID NO.: 1); reverse primers: 5′-AGGCACATTTAAATCCATGATTACGCCAAGCT) (SEQ ID NO.: 2). The pE2FGBV siRNA^{ras} shuttle plasmid was generated through restriction digestion by ClaI/SwaI and re-ligation, (20). This 157 bp expression cassette was inserted to replace the 962 bp E3B deleted region of ONYX-411, corresponding to 29,859-30,820 nt sequence of the wt Ad5 genome. The restriction enzyme-digested pE2FGBV siRNA^{ras-4} (by EcoR I and Bam HI) and ONYX-411 DNA (by EcoR I) were ligated with T4 DNA ligase for 18 hrs at 23° C., and transfected into permissive 293 cells (FuGENE 6 Transfection Reagent, Roche). The clonally selected ONYX-411-siRNA^{ras-4} virus was verified by PCR, using primers specific for the E3B region insertion sites (forward primers: 5′-CACATTGGCTGCGGTTTCTCACAT (SEQ ID NO.: 3); reverse primer: 5′-CGCGCTTCATCTGCAACAACATGA(SEQ ID NO.: 4)) and bidirectional DNA sequencing (ABI 310 Genetic Analyzer, Applied Biosystems, Foster City, Calif.). Titer of the cesium chloride-purified virus was determined by plaque forming assays with 293 cells.

RT-PCR. Total RNAs from cultures (4×10⁵ cells/well) were isolated (RNeasy kit, QIAGEN), and 1 μg of total RNA from each sample was co-amplified with previously described primers specific to K-ras or β-actin internal control genes by RT-PCR (SuperScript one-step RT-PCR, Invitrogen) (52° C., 30 min; 94 C, 2 min; then 32 cycles at 94° C., 30 sec; 55° C., 30 sec; 72° C., 30 sec) (18). Amplification products of the expected size were quantified by densitometer measurements and normalized to β-actin values, which serve as internal reference in this semi-quantitative RT-PCR analysis (Alphalmager 2000 D, Alpha Innotech; NIH/Scion Image Software, Scion, Frederick, Md.) (18). A minimum of three determinations was used to establish mean (±SEM) OD measurements.

Viral Yield Quantification. To determine the viral yield in infected cultures (1×10⁵ cells/well, 0.1 MOI), cells were collected at 4 hrs, then at graded time points post-infection. Cellular DNA from cell pellets or supernatants was isolated (QIAamp DNA blood kit, Qiagen) and quantified by OD₂₆₀/OD₂₈₀ measurements. Real time PCR (TaqMan® Universal PCR Master Mix, Applied Biosystems) was performed using 0.1 ug of DNA and primers probe specific to the adenoviral late hexon (forward primer: 5′-TGCCTTTAC-GCCACCTTCTTC (SEQ ID NO.: 5); reverse primer: 5′-CGGGTATAG-GGTAGAGCATGTTG (SEQ ID NO.: 6); FAM/BHQ labeled standard probe: 5′-CCACAACACCGCCTCCACGCTTGA (SEQ ID NO.: 7); Biosearch Technologies, Novato, Calif.). Parallel reactions using DNA recovered from a pre-determined copy number of wild type Ad5 virus is used as internal reference controls. Viral yield (viral particles/1×10⁵ cells) at different time points post-viral infection was normalized to the input dose determined in the same manner at 4 hrs post-initial infection.

Proliferative Assay. Proliferative activity of tumor cells following cotransfection with siRNA and ONYX-411 was determined by a bromodeoxyuridine (BrdU) incorporation assay. At 24 hrs post-viral infection, (1×10⁵ cells/well in a 24-well plate), H441 cells were transfected with K-ras siRNA-2, -4, -5 or control none-silencing siRNA (150 nM; Gene Silencing transfection reagent). The cells were harvested after 24 hrs, collected after trypsin/EDTA treatment, and suspended in 1.0 ml culture medium (RPMI1640+10% FBS). 50 ul of the cell suspension (approximate 5×10³ cells/well) were inoculated in triplicates into a 96-well plate containing 150 ul of culture medium, and cultured for another 48 hrs. BrdU was added according to manufacturer's protocol (R&D System). Wells seeded with graded numbers (10³ to 10⁵) of untreated cells were used to establish the linear relationship of cell number vs. adsorbance as a function of BrDU uptake (SpectraMax 340, Molecular Device, Sunnyvale, Calif.). A minimum of three experiments was conducted to establish mean±SD values for each treatment.

Cell Viability Analysis. Total viable cells of A549 and H79 cultures were determined daily after transfection with siRNAs or viral infection by the standard trypan blue exclusion analysis by enumerating 2-500 total cells. For determination of cytotoxicity to HMEC cells, the proportion of non-viable cells as identified by morphological features (rounding and increased granularity) was determined by light microscopic enumeration of two 200× magnification fields (approximately 200 cells per field). Value represents mean of two field enumerations for each treatment.

MTT Assay. Mitochondria metabolic activity was quantified by the reduction of 3-[4,5-dimethlythiazol-2-yl]-2,5-diphenyltetrazolium bromide-(MTT) by metabolically active cells to insoluble purple formazan dye crystals. To this end, target cells were inoculated in wells of a 96-well plate (1×10⁴ cells/well), then infected with wt dl309, or the recombinant adenovirus to be tested (0.1 to 25 MOI) for 72 to 120 hrs. MTT (R&D Systems, Minneapolis, Minn.) was added according to manufacturer's protocol. The colorimetric reaction was quantified by spectrophotometric determinations at 570 nm, with 690 nm as reference (SpectraMax 340, Molecular Device), For each experiment, OD measurements of wells seeded with graded numbers of untreated cells (2.5×10³, 5×10³, 1×10⁴ and 2×10⁴) were used to generate a standard curve for extrapolation of equivalent viable cell numbers in virus-treated and untreated cultures. A minimum of three replicates in each study were carried out to determine mean numbers of viable cells in treated and untreated cultures

Determination of Anti-tumor Activity with Human Tumor Xenograft in nu/nu Mice. H79 cells (3×10⁶ cells in 0.1 ml of PBS) were inoculated subcutaneously into the right flank of athymic nu/nu mice (5-6 weeks old, Harlan/Sprague). Tumor growth was closely monitored. Treatment with viral constructs was initiated when tumor xenografts reached a size of ≧80 mm³. Each mouse received daily intratumoral Injections of Internavec, ONYX-411, or ONYX-411-siRNA^{GFP} at 1×10⁷ or 1×10⁸ pfu (in volume of 0.1 ml) for 5 consecutive days. Control group mice received the same number of injections with PBS. Xenograft sizes (width and length) were measured by a vernier caliper twice a week. Tumor volume (V) was calculated by the formula: V=(L)(W²)/2 (18). Animals were euthanized when tumor xenograft exceeded 2 cm in diameter or at 60 days post-injection, whichever came first. Mean differences of tumor volume among treatment groups at a given time points were analyzed statistically by the two-tailed student's t test. All procedures involving animal use were approved by the Institutional Animal Care and Use Committee at Baylor University Medical Center.

Cell Cycle and Apoptotic Analysis. H79 cells (4×10⁵/well) in triplicate at a given point in 6-well plates were infected with Internavec, ONYX-411 and dl309 virus at 5 MOI, and collected at the time of 48 hrs, 72 hrs or 96 hrs post-infection. The DNA content was determined by propidium iodine (PI, Sigma, St. Louis, Mo.) and flow cytometric analyses. Briefly, cells were fixed in 80% ethanol in 1×PBS at −20° C. overnight prior to PI staining. 1×10⁶ cells each sample were stained with 1.0 ml of 1×PBS containing PI (40 μg/ml) and RNase A (Qiagen, 100 μg/ml) at 37° C. for 30 min. Flow cytometric analysis of PI staining cells was immediately carried out with a sampling size of 20,000 cells (FACScan, Becton Dickinson). The obtained DNA content distributions were transferred into ModFit computer software (Verity, Inc; Topsham, Me.). The percentage of cell population at each cell cycle phase was determined by its relative area under the curve of width versus area of the fluorescence histogram. For analysis of apoptotic activity, H79 cells were infected with the viral constructs (5 MOI) or Fas ligand as positive control (0.4 μg/ml, Beckman Coulter). Apoptotic cells were quantified by flow cytometric analysis as a function of 7-amino actinomycin D (7-AAD, Invitrogen) incorporation (100 uM, 20 min, on ice, in PBS with 1% FBS) at 72 hrs post-infection as described previously (18). The reactants were fixed paraformaldehyde (1%). Analysis was carried out at the emitted wavelength of >650 nm following laser excitation at 488 nm, based on 1×10⁴ acquired events (FACScan, Becton Dickinson).

cDNA Microarray Analysis. Total RNA was isolated from H79 cultures with the RNeasy kit (Qiagen) at 36 hrs following infection with Internavec, parental ONYX-411 or the control viral construct containing an siRNA against the GFP reporter gene (ONYX-411-siRNA^{GFP}) (5 MOI, 4×10⁵ cells/well in a 6-well plate). Gene expression profiles were determined by array hybridization reactions with the Human Genome U133 Plus 2.0 oligonucleotide probe array containing gene probes from 38,500 human genes (Affymetrix, Santa Clara, Calif.) according to standard protocols provided by the manufacturer (Microarray Core Facility, University of Texas Southwestern Medical Center, Dallas, Tex.). An absolute expression analysis was carried out by using Affymetrix MAS 5.0, and the data were imported into Gene Spring 7.2 software (Silicon Genetics, Santa Clara, Calif.) for differential subtraction for hybridization reactions with a minimum signal intensity of ≧50 to identify transcripts that differed by a magnitude of ≧2-fold, and compared by hierarchical clustering analysis.

Selection of K-ras-reactive siRNA. siRNA^{ras}s that demonstrated significant K-ras knockdown activity include oligonucleotides that target the consensus sequence upstream of codon 12 (siRNA^{ras-2}) as well as codon 12 sequences that are unique to ras^{v12} (siRNA^{ras-4}) or wild type (ras^{g12}; siRNA^{ras-5}) phenotype (FIG. 1a). siRNA^{ras-2}, siRNA^{ras-4}, and siRNA^{ras-5} reduced K-ras mRNA expression of the heterozygous H441 cells (K-ras^{g12/v12}) by 49.2%±5.6%, 41.4%±17.8% and 36.3%±14.6%, respectively, at 22-24 hrs post-transfection (FIG. 1b). Control siRNA did not significantly affect K-ras mRNA level. siRNA^{ras-2}, with its consensus binding capacity to both wild type and mutant K-ras mRNA, is predisposed to a higher knockdown activity than siRNA^{ras-4} or siRNA^{ras-5}. However, an increased knockdown efficiency was not evident, probably due to closer proximity of the targeted site in reference to the K-ras exon 1 starting codon (reviewed in 5). Based on evaluations with siRNA^{ras}-2, K-ras knockdown was dose- (50 nM: 35.3±9.9%; 100 nM: 41.1%; 150 nM: 67.9±23.8%) and time-dependent, with optimal knockdown with 150 nM at 20-24 hrs.

The antitumor activity of siRNA^{ras} was examined with H441 cells with the heterozygous K-ras^{g12/v12} phenotype, based on BrdU incorporation at 72 h (FIG. 1c). The extent of growth reduction (siRNA^{ras-4}: 52%; si-RNA^{ras-2}: 36%; p=0.05, n=3) appeared proportionate to K-ras knockdown in this limited analyses. With a mean lipofection efficiency was 48%, our findings of a comparable level of target mRNA knockdown and growth reduction are indicative of the effectiveness of siRNA treatment.

RAS-knockdown enhanced tumor growth inhibition without aborting viral replication. To consider the impact of RAS knockdown on viral replication, qPCR analysis was carried out on ONYX-411+siRNA^{ras-2} treated A549 cells (K-ras^{s12/s12}), using primers specific to the adenovirus late hexon gene. Viral yield did not differ significantly from cells infected with parental ONYX-411 (FIG. 1d), indicating that “late phase” knockdown of K-ras (at 24 hrs post-viral infection) did not adversely affect viral replication (FIG. 1d). A limitation of this model is an incomplete overlap between siRNA-transfected and viral-infected cells (efficiency of ≈50% for either treatment). Nonetheless, the observed additive cytotoxic effect supports the premise that late phase RAS knockdown enhances viral oncolysis without adversely altering viral replication.

Limited Cytotoxicity of ONYX-411-siRNA^{ras-4} to non-malignant cells. ONYX-411 virus has been previously shown having a significantly lower toxicity to normal cells (21). To define whether arming K-ras siRNA affect its toxicity profile to normal cells, we examined cytopathic effect of human mammary epithelium cells (HMEC) post-infection by light microscopic analysis. Internavec behaved similarly as ONYX-411 virus, and produced minimal toxicity to normal HMEC cells at day 5 post-infection (FIG. 3). By comparison, wt adenovirus-infected cells showed almost 100% cytopathic effect at day 5 post-infection. The comparable toxicity level between Internavec and ONYX-411 virus were also confirmed by MTT assay (data not shown). These findings suggest that K-ras siRNA arming did not markedly affect the cytotoxicity profile of the parental ONYX-411 virus to normal cells.

Gene Array analysis of Internavec treatment. Gene expression array analysis was carried out on the total RNA of H79 cells untreated or treated with ONYX-411, ONYX-411-siRNA^{GFP} and Internavec to explore the molecular mechanisms responsible for the enhanced anti-tumorigenesis of Internavec. Following normalization to hybridization signal by the untreated culture, differentiation expression analysis was carried out with Internavec culture against ONYX-411 and ONYX-411-siRNA^{GFP} cultures, using Affymetrix and David 2.0 softwares (NIH). 198 genes were uniquely downregulated following Internavec treatment, and 234 were upregulated by ≧2-fold. 9 of the 198 genes were known to be associated with the Ras signaling pathway, including 4 that have been known to regulate the cell cycle pathway (FIG. 5c). 41 genes were assigned to pathways related to cell cycle, cell-cell interaction and signal transduction, RNA and protein metabolism, stress and immune response (Table 1).

TABLE 1
cDNA gene expression array analysis after Internavec treatment^{a}.

| Affected | | Relative | |
|---|---|---|---|
| Pathways | Downregulated Genes | Expression | Possible Outcome of Downregulation |
| Cell cycle | Insulin receptor substrate 4 | 0.34 | Disrupts Shp2 and Grb2 |
| and | (IRS4) | | phosphorylation, blocking Ras, PI3K and |
| Proliferation | | | AKT/PKβ activites. |
| | Protein kinase β (AKT2) | 0.38 | Inhibits activation of NFκB and cell |
| | | | survival. |
| | Cyclic GMP inhibited | 0.40 | Induces apoptosis. |
| | phosphodiesterase A | | |
| | (PDE3) | | |
| | Noggin precursor (NOG) | 0.40 | Activates pro-apoptotic TGFβ pathway. |
| | Glycogen synthase kinase-3 | 0.43 | TNF-related apoptosis in myc- |
| | β (GSK3β) | | overexpressing cancer cells. |
| | Transcription factor E2F2 | 0.45 | ↓ G1→S phase entry. |
| | (E2F2) | | |
| | Ponsin (SORBS1) | 0.45 | ↓ growth and survival signals. |
| | Transcription cofactor | 0.47 | ↓ cell growth/differentiation and |
| | vestigial-like protein 1 | | tumorigenesis. |
| | (VGLL1) | | |
| | Sodium/potassium- | 0.5 | ↓ cell proliferation. |
| | transporting ATPase alpha- | | |
| | 4 chain (ATP1A4) | | |
| Cell-cell | Glucocorticoid receptor | 0.10 | ↓ Ras activity |
| interaction | DNA binding factor 1 | | |
| and signal | (GRLF-1) | | |
| transduction | Integrin α8 (ITGA8) | 0.12 | ↓ tumor malignancy and cell migration |
| | Glutamine | 0.18 | ↑ cytotoxicity to cancer cells. |
| | phosphoribosylpyrophospha | | |
| | te amidotransferase (PPAT) | | |
| | Esophagus cancer-related | 0.19 | ↑ neoplasia. |
| | gene 2 (ECG2) | | |
| | Epidermal growth factor | 0.21 | Induces apoptosis and inhibits cell |
| | receptor (EGFR) | | proliferation. |
| | Coactivator for steroid | 0.21 | Curtails interaction with steroid receptors |
| | receptor (ZNF451) | | involved in turmorigenesis. |
| | Atrophin-1 interacting | 0.29 | ↑ vascularization and angiogenesis. |
| | protein 3 (BAIAP1) | | |
| | Discoidin (DCBLD1) | 0.35 | ↓ cell survival possibly via the functions |
| | | | of decreased angiogenesis, motility and |
| | | | invasion. |
| | Transducin (beta)-like 2 | 0.38 | ↓ proliferation and survival in cancer |
| | (TBL2) | | cells. |
| | CD90 (THY1) | 0.39 | ↓ lymphocyte stimulation. |
| | Fibrillin-like protein 1 | 0.40 | ↓ MBP1 (mutant p53-binding protein 1) |
| | (EFEMP1) | | activity and tumor cell growth. |
| | G protein-coupled receptor | 0.42 | ↓ tumorigenesis and metastasis. |
| | 153 (GPR153) | | |
| | Sialic acid binding Ig-like | 0.44 | ↓ expression of tumor-associated gene. |
| | lectin 8 (SIGLEC8) | | |
| | Tracheo-bronchial mucin 5 | 0.45 | ↓ cell growth and tumor invasive |
| | (MUC5AC) | | activities. |
| | Phospholipase C like-1 | 0.45 | ↓ tumor progression. |
| | (PLCL1) | | |
| | Fused in myeloproliferative | 0.46 | Downregulation of this tyrosine kinase |
| | disorders protein (ZNF198) | | prevents oncogenic protein Bcr-Abl |
| | | | activity. |
| | Cancer-associated | 0.47 | ↓ malignancy and tumor progression. |
| | nucleoprotein (CANP) | | |
| | Erbb2-interacting protein | 0.47 | Induces apoptosis and inhibits cell |
| | (ERBB2IP) | | proliferation. |
| | Mitogen-activated protein | 0.48 | Promotes apoptosis. |
| | kinase kinase kinase 3 | | |
| | (MAP3K3) | | |
| | MAP4K5 | 0.50 | Promotes apoptosis. |
| | Hematopoietic zinc finger | 0.50 | Checkpoint control and cell survival. |
| | protein (ZNF385) | | |
| RNA | Transcription factor 17 | 0.48 | Decreased nucleolar disruption and |
| metabolism | (ZNF354A) | | transcriptional repression. |
| Protein | Ubiquitin-conjugating | 0.23 | ↑ apoptosis. |
| metabolism | enzyme E2 E2 (UBE2E2) | | |
| | UBE2D3 | 0.33 | ↑ apoptosis. |
| | HECW2 | 0.38 | Destabilizes the proapoptotic regulator |
| | | | p73. |
| | Hect domain and RLD4 | 0.48 | ↓ active G proteins. |
| | (HERC4) | | |
| Stress and | Pre-B cell growth | 0.38 | ↓ metastasis. |
| immune | stimulating factor | | |
| response | (CXCL12) | | |
| | Melanoma-differentiation | 0.39 | Reduced tumor growth regulatory and |
| | associated protein 7 (IL24) | | immune-stimulatory activities. |
| | p43 (SCYE1) | 0.43 | Reduced mitochondria function and |
| | | | malignancy transforming activities. |
| | Colony-stimulating factor 2 | 0.44 | ↓ cell proliferation and growth. |
| | (CSF2) | | |
| | Lymphotoxin β (LTB) | 0.45 | Inhibits angiogenesis. |
| | Lymphocyte activation | 0.48 | ↓ NFκB transcription and Bcl-2 |
| | antigen CD30 (TNFRSF8) | | activation, with ↑ apoptosis. |

^{a}Affymetrix Human Genome U133 Plus 2.0 cDNA microarray analysis was carried out with total RNA from H79 cells that were untreated or treated with ONYX-411, ONYX-411-siRNA^{GFP} and Internavec, followed by Affymetrix and David 2.0 software (NIH) analysis for pathway assignments. 41 of 198 uniquely downregulated (≧2-fold) following Internavec treatment were summarized.
^{b}Ratio of the signal intensity of Internave treatment divided by the signal intensity of untreated culture in a given gene, as detected by microarray analysis.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

In the claims, all transitional phrases such as “comprising,” “including, ” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

1. Elbashir, S. M., Harborth, J., Lendeckel, W., Yalcin, A., Weber, K., Tuschl, T. Duplexes of 21-nucleotide RNAs mediate RNA interference in cultured mammalian cells. Nature. 411(6836):494-8 (2001).

2. Lieberman, J., Song, E., Lee, S. K., Shankar, P. Interfering with disease: opportunities and roadblocks to harnessing RNA interference. Trends Mol Med 9: 397-403 (2003).

3. Jackson, A. L., Bartz, S. R., Schetlter, J., Kobayashi, S. V., Burchard, J., Mao, M., Li, B., Cavet, G., Linsely, P. S. Expression profiling reveals off-target gene regulation by RNA. Nat. Biotechnol. 21: 635-7(2003).

4. Napoli, C., Lemieux, C., Jorgensen, R. Introduction of a chimeric chalcone synthase gene into petunia results in reversible co-suppression of homologous genes in trans. Plant Cell 2: 279-89 (1990).

5. Tong, A. W., Zhang, Y. A., Nemunaitis, J. Small interfering RNA for experimental therapy (review). Curr. Opin. Mol. Therap. 7:114-24 (2005).

6. Novina, C. D., Sharp, P. A.: The RNAi revolution. Nature 430: 161-4 (2004).

7. Brummelkamp, T. R., Bernards, R., Agami, R. Stable suppression of tumorigenicity by virus-mediated RNA interference. Cancer Cell. 2: 243-7. (2002).

8. Kawasaki, H., Taira, K. Short hairpin type of dsRNAs that are controlled by tRNA(Val) promoter significantly induce RNAi-mediated gene silencing in the cytoplasm of human cells. Nucleic Acids Res. 31 (2): 700-7 (2003).

9. McCaffrey, A. P., Meuse, L., Pham, T. T., Conklin, D. S., Hannon, G. J., Kay, M. A. RNA interference in adult mice. Nature 418: 38-9 (2002).

10. Sorensen, D. R., Leirdal, M., Sioud, M. Gene silencing by systemic delivery of synthetic siRNAs in adult mice. J. Mol. Biol. 327: 761-6 (2003).

11. Xia, H., Mao, Q., Paulson, H. L., Davidson, B. L. siRNA-mediated gene silencing in vitro and in vivo. Nat. Biotechnol. 20 (10):1006-10 (2002).

12. Zhang, Y., Zhang, Y. F., Bryant, J., Charles, A., Boado, R. J., Pardridge, W. M. Intravenous RNA interference gene therapy targeting the human epidermal growth factor receptor prolongs survival in intracranial brain cancer. Clin. Cancer Res. 10: 3667-77 (2004).

13. Martuza, R. L., Malick, A., Markert, J. M., Ruffner, K. L., Coen, D. M. Experimental therapy of human glioma by means of a genetically engineered virus mutant. Science. 252(5007): 854-6 (1991).

14. Toth, K., Doronin, K., Tollefson, A. E., Wold. W. S. M. A multitasking oncolytic adenovirus vector. Mol. Therapy 7: 435-437 (2003).

15. Lin, E., Nemunaitis, J. Oncolytic viral therapies. Cancer Gene Therapy 11: 643-64 (2004).

16. Kasuya, H., Takeda, S., Nomot, S., Nakao, A. The potential of oncolytic virus therapy for pancreatic cancer. Cancer Gene Therapy 12: 725-36 (2005).

17. Tong, A. W. Oncolytic viral therapy for human cancers: Challenges revisited (review). Drug Dev. Res. 66: 1-18 (2006).

18. Zhang, Y. A., Nemunaitis, J., Scanlon, K. J., Tong, A. W. Anti-tumorigenic effect of a K-ras ribozyme against human lung cancer cell line heterotransplants in nude mice. Gene Ther. 27 (23): 2041-50 (2000).

19. Doronin, K., Toth, K., Kuppuswamy, M., et al. Tumor-specific, replication-competent adenovirus vectors overexpressing the adenovirus death protein. J. Virol. 74: 6147-55 (2000).

20. Zhan, J., Gao, Y., Wang, W., Shen, A., Aspelund, A., Young, M., Laquerre, S., Post L., Shen, Y. Tumor-specific intravenous gene delivery using oncolytic adenoviruses. Cancer Gene Ther. 12:19-25 (2005).

21. Johnson, L., Shen, A., Boyle, Kunich, J., Pandey, K., Lemmon, M., Hermiston, T., Giedlin, M., McCormick, F., Fattaey, A. Selectively replicating adenoviruses targeting deregulated E2F activity are potent, systemic antitumor agents. Cancer Cell. 4: 325-37 (2002).

22. Carette, J. E., Overmeer, R. M., Schagen, F. H. E., Alemany, R., Barski, O. A., Gerritsen, W. R., van Beuschem, V. W. Conditionally replicating adenoviruses expressing short hairpin RNAs. Cancer Res. 64: 2663-7 (2004)

23. Vaillancourt, M. T., Atencio, I., Quijano, E., Howe, J. A., Ramachandra, M. Inefficient killing of quiescent human epithelial cells by replicating adenoviruses: potential implications for their use as oncolytic agents. Cancer Gene Ther. 12: 691-8 (2005).

24. Zhao, H., Granberg, F., Elfineh, L., Pettersson, U., Svensson, C. Strategic attack on host cell gene expression during adenovirus infection. J. Virol. 77: 11006-11015 (2003).

25. Berk, A. J. Recent lessons in gene expression, cell cycle control, and cell biology from adenovirus. Oncogene 24: 7673-85 (2005).

26. Fan, J., Bertino J. R. K-ras modulates the cell cycle via both positive and negative reegulatory pathways. Oncogene 14: 2595-607 (1997).

27. Hitomia, M., Stacey, D. W. Cellular Ras and cyclin D1 are required during different cell cycle periods in cycling NIH 3T3 cells. Mol. Cell. Biol. 19: 4623-32 (1999).

28. Fink, S. L., Cookson, B. T. Apoptosis, pyroptosis, and necrosis: Mechanistic description of dead and dying eukaryotic cells. Infect. Immun. 73: 1907-1916 (2005).

29. Jian, K., Coppola, D., Crespo, N. C., Nicosia, S. V., Hamilton, A. D., Sebti, S. M., Cheng, J. Q. The phosphoinostide 3-OH kinase/AKT2 pathway as a critical target for famesyltransferase inhibitor-induced apoptosis. Mol. Cell Biol. 20: 139-48 (2000).

30. Andersson, M. G., Joost Haasnoot, P. C., Xu, N., Berenjian, A., Berhout, B., & Akusjarvi, G. Suppression of RNA interference by adenovirus virus-associated RNA. J. Virol. 79, 9556-65 (2005)

What is claimed is:

1. A gene delivery vector comprising a replication-competent, oncolytic adenovirus that expresses a siRNA.

2. The vector of claim 1, wherein the target cell for the vector is a human cancer cell.

3. The vector of claim 1, wherein the adenovirus comprises an ONYX virus.

4. The vector of claim 1, wherein the adenovirus comprises an ONYX-411, ONYX-200 or an ONYX-015 virus, or other similarly modified replication-competent, oncolytic viruses including AdΔ24.

5. The vector of claim 1, wherein the siRNA targets a gene that encodes an oncogene, a transcription factor, a receptor, an enzyme, a structural protein, a cytokine, a cytokine receptor, a lectin, a selectin, an immunoglobulin, a kinase and a phosphatase.

6. The vector of claim 1, wherein the siRNA against the K-ras oncogene alone kills at least 35% of the target cells.

7. The vector of claim 1, wherein the siRNA against K-ras oncogene and the vector kills at least 50% of the target cells in 72 hours.

8. The vector of claim 1, wherein the siRNA comprises an oncogene K-ras^{v12}-specific siRNA^{ras-4} hairpin insert.

9. The vector of claim 1, wherein the siRNA targets a gene that encodes a protein selected from the group consisting of amyloid protein, amyloid precursor protein, angiostatin, endostatin, METH-1, METH-2, Factor IX, Factor VIII, collagen, cyclin dependent kinase, cyclin D1, cyclin E, WAF 1, cdk4 inhibitor, MTS 1, cystic fibrosis transmembrane conductance regulator, IL-1, IL-2, IL-3, IL-4, IL-5, IL-6, IL-7, IL-8, IL-9, IL-10, IL-11, IL-12, IL-13, IL-14, IL-15, IL-16, IL-17, erythropoietin, G-CSF, GM-CSF, M-CSF, SCF, thrombopoietin, BNDF, BMP, GGRP, EGF, FGF, GDNF, GGF, HGF, IGF-1, IGF-2, KGF, myotrophin, NGF, OSM, PDGF, somatotrophin, TGF-β, TGF-α, VEGF, interferon, TNF-.alpha., TNF-.beta., cathepsin K, cytochrome p-450, farnesyl transferase, glutathione-s transferase, heparanase, HMG CoA synthetase, n-acetyltransferase, phenylalanine hydroxylase, phosphodiesterase, ras carboxyl-terminal protease, telomerase, TNF converting enzyme, E-cadherin, N-cadherin, selectin, CD40, 5-α reductase, atrial natriuretic factor, calcitonin, corticotrophin releasing factor, glucagon, gonadotropin, gonadotropin releasing hormone, growth hormone, growth hormone releasing factor, somatotropin, insulin, leptin, luteinizing hormone, luteinizing hormone releasing hormone, parathyroid hormone, thyroid hormone, thyroid stimulating hormone, immunoglobulin, CTLA4, hemagglutinin, major histocompatibility factor (MHC), VLA-4, kallikrein-kininogen-kinin system, CD4, sis, hst, ras, abl, mos, myc, fos, jun, H-ras, ki-ras, c-fms, bcl-2, L-myc, c-myc, gip, gsp, HER-2, bombesin receptor, estrogen receptor, GABA receptor, EGFR, PDGFR, FGFR, NGFR, GTP-binding regulatory proteins, interleukin receptors, ion channel receptors, leukotriene receptor antagonists, lipoprotein receptors, opioid pain receptors, substance P receptors, retinoic acid and retinoid receptors, steroid receptors, T-cell receptors, thyroid hormone receptors, TNF receptors, tissue plasminogen activator; transmembrane receptors, calcium pump, proton pump, Na/Ca exchanger, MRP 1, MRP2, P170, LRP, cMOAT, transferrin, APC, brca1, brca2, DCC, MCC, MTS1, NF1, NF2, nm23, p53 and Rb.

10. A vector comprising a replication-competent, oncolytic adenovirus comprising an siRNA that mediates siRNA-mediated oncogene knockdown and viral oncolysis.

11. The vector of claim 10, wherein the adenovirus comprises an ONYX-411, ONYX-200, ONYX-015, or Ad5Δ24 virus.

12. The vector of claim 10, wherein the siRNA targets a gene that encodes an oncogene, a transcription factor, a receptor, an enzyme, a structural protein, an cytokine, a receptor, a cytokine receptor, a lectin, a selectin, an immunoglobulin, a kinase and a phosphatase.

13. The vector of claim 10, wherein the siRNA targets one or more oncogene or tumor suppressor gene selected from bcl-2, bcr-ab1, bek, BPV, c-abl, c-fes, c-fms, c-fos, c-H-ras, c-kit, c-myb, c-myc, c-mos, c-sea, cerbB, DCC, erbA, erbB-2, ets, fig, FSFV gp55, Ha-ras, HIV tat, HTLV-1 tat, JCV early, jun, L-myc, lck, LPV early, met, N-myc, NF-1, N-ras, neu, p53, Py mTag, pim-1, ras, RB, rel, retinoblastoma-1, SV-40 Tag, TGF-α, TGF-β, trk, trkB, v-abl, v-H-ras, v-jun, or WT-1.

14. The vector of claim 10, wherein the siRNA comprises K-ras oncogene and the vector kill at least 35% of the target cells.

15. The vector of claim 10, wherein the siRNA comprises an oncogene K-ras^{v12}-specific siRNA^{ras-4} hairpin insert.

16. The vector of claim 10, wherein a target gene of the siRNA is p53, p16, p21, MMAC1, p73, zac1, C-CAM, BRCA1, Rb, Harakiri, Ad E1 B, ICE-CED3 protease, IL-2, IL-3, IL-4, IL-5, IL-6, IL-7, IL-8, IL-9, IL-10, IL-11, IL-12, IL-13, IL-14, IL-15, TNF, GMCSF, α-interferon, β-interferon, γ-interferon, CFTR, EGFR, VEGFR, IL-2 receptor and the estrogen receptor, Bcl-2 gene family is Bcl-2 or Bcl-xL, ras, myc, neu, raf erb, src, fins, jun, trk, ret, gsp, hst, and abl.

17. A method of modulating gene expression comprising:
contacting a target cell with a vector comprising a replication-competent, oncolytic adenovirus comprising an siRNA that mediates siRNA-mediated oncogene knockdown and viral oncolysis.

18. The method of claim 17, wherein the adenovirus comprises an ONYX-411, ONYX-200, ONYX-015, or Ad5Δ24 virus.

19. The method of claim 17, wherein the siRNA targets a gene that encodes an oncogene, a transcription factor, a receptor, an enzyme, a structural protein, an amyloid protein, amyloid precursor protein, angiostatin, endostatin, METH-1, METH-2, Factor IX, Factor VIII, collagen, cyclin dependent kinase, cyclin D1, cyclin E, WAF 1, cdk4 inhibitor, MTS1, cystic fibrosis transmembrane conductance regulator, IL-1, IL-2, IL-3, IL-4, IL-5, IL-6, IL-7, IL-8, IL-9, IL-10, IL-11, IL-12, IL-13, IL-14, IL-15, IL-16, IL-17, erythropoietin, G-CSF, GM-CSF, M-CSF, SCF, thrombopoietin, BNDF, BMP, GGRP, EGF, FGF, GDNF, GGF, HGF, IGF-1, IGF-2, KGF, myotrophin, NGF, OSM, PDGF, somatotrophin, TGF-β, TGF-α, VEGF, interferon, TNF-.alpha., TNF-.beta., cathepsin K, cytochrome p-450, farnesyl transferase, glutathione-s transferase, heparanase, HMG CoA synthetase, n-acetyltransferase, phenylalanine hydroxylase, phosphodiesterase, ras carboxyl-terminal protease, telomerase, TNF converting enzyme, E-cadherin, N-cadherin, selectin, CD40, 5-α reductase, atrial natriuretic factor, calcitonin, corticotrophin releasing factor, glucagon, gonadotropin, gonadotropin releasing hormone, growth hormone, growth hormone releasing factor, somatotropin, insulin, leptin, luteinizing hormone, luteinizing hormone releasing hormone, parathyroid hormone, thyroid hormone, thyroid stimulating hormone, immunoglobulin, CTLA4, hemagglutinin, major histocompatibility factor (MHC), VLA-4, kallikrein-kininogen-kinin system, CD4, sis, hst, ras, abl, mos, myc, fos, jun, H-ras, ki-ras, c-fins, bcl-2, L-myc, c-myc, gip, gsp, HER-2, bombesin receptor, estrogen receptor, GABA receptor, EGFR, PDGFR, FGFR, NGFR, GTP-binding regulatory proteins, interleukin receptors, ion channel receptors, leukotriene receptor antagonists, lipoprotein receptors, opioid pain receptors, substance P receptors, retinoic acid and retinoid receptors, steroid receptors, T-cell receptors, thyroid hormone receptors, TNF receptors, tissue plasminogen activator; transmembrane receptors, calcium pump, proton pump, Na/Ca exchanger, MRP 1, MRP2, P170, LRP, cMOAT, transferrin, APC, brca1, brca2, DCC, MCC, MTS1, NF1, NF2, or nm23.

20. The method of claim 17, wherein the siRNA targets a Ras protein, p53, pRb, EF2-1, bcl-2, bcr-abl, bek, BPV, c-abl, c-fes, c-fms, c-fos, c-H-ras, c-kit, c-myb, c-myc, c-mos, c-sea, cerbB, DCC, erbA, erbB-2, ets, fig, FSFV gp55, Ha-ras, HIV tat, HTLV-1 tat, JCV early, jun, L-myc, lck, LPV early, met, N-myc, NF-1, N-ras, neu, p53, Py mTag, pim-1, ras, RB, rel, retinoblastoma-1, SV-40 Tag, TGF-α, TGF-β, trk, trkB, v-abl, v-H-ras, v-jun, or WT-1.

21. The method of claim 17, wherein the siRNA that targets the K-ras oncogene kills at least 35% of the target cells.

22. The method of claim 17, wherein the siRNA comprises an oncogene K-ras^{v12}-specific siRNA^{ras-4} hairpin insert.

23. A method of treating a patient in need of gene therapy comprising:
identifying one or more target cells that are in need of gene therapy; and
making a replication-competent, oncolytic adenovirus comprising an siRNA that mediates siRNA-mediated gene modulation; and
contacting the target cell with the adenovirus, wherein at least 35% of the target cells are killed using the siRNA expressed by the replication-competent, oncolytic adenovirus.
